(19) United States
(12) Patent Application Publication
(10) Pub. No.: US 2022/0142845 A1
(43) Pub. Date: May 12, 2022

(54) TELESCOPING LONGITUDINAL FEMORAL SUPPORT SYSTEMS

(71) Applicant: INNOVATIVE ORTHOPEDIC TECHNOLOGIES, IOT AG, Baar (CH)

(72) Inventors: Michael Vincent Jekot, Austin, TX (US); Stefan Kreuzer, Houston, TX (US); Manfred Menzi, Buchs (CH)

(73) Assignee: INNOVATIVE ORTHOPEDIC TECHNOLOGIES, IOT AG, Baar (CH)

(21) Appl. No.: 17/525,385

(22) Filed: Nov. 12, 2021

Related U.S. Application Data
(60) Provisional application No. 63/113,169, filed on Nov. 12, 2020.

(51) Int. Cl. A61G 13/12; A61B 5/00

(52) U.S. Cl. CPC A61G 13/129; A61G 13/1245; A61B 5/702

(57) ABSTRACT

A telescopic assembly for supporting a patient's femur includes an elongate tubular base configured to be fixably attached to a table. The tubular base has a longitudinal axis. The telescopic assembly also includes an elongate extension member slidably disposed in the tubular base and configured to telescopically extend longitudinally from the tubular base. Further, the telescopic assembly includes a femur support member coupled to an end of the extension member and configured to be positioned below and support the femur of the patient. The femur support member is configured to move longitudinally with the extension member relative to the tubular base.

### CROSS-REFERENCE TO RELATED APPLICATIONS

This application claims benefit of U.S. provisional patent application Ser. No. 63/113,169 filed Nov. 12, 2020, and entitled “Telescoping Longitudinal Femoral Support Systems,” which is hereby incorporated herein by reference in its entirety for all purposes.

### STATEMENT REGARDING FEDERALLY SPONSORED RESEARCH OR DEVELOPMENT

Not applicable.

### BACKGROUND

This disclosure relates generally to devices and methods for supporting and manipulating a patient's leg during surgery or diagnostic procedures. More specifically, this disclosure relates to devices and methods for supporting and manipulating a patient's thigh and femur during surgery (e.g., hip joint surgery) or for diagnostic analysis of the leg (e.g. x-ray).

During surgery on a patient's leg (e.g., hip surgery), certain positions and orientations of the leg may be preferred by the surgeon. For example, during one phase of direct anterior hip replacement surgery, the surgeon may want to orient and support the patient's thigh and femur at a position and orientation that provides optimal or near optimal femoral exposure.

### BRIEF SUMMARY OF THE DISCLOSURE

Embodiments of telescopic assemblies for supporting a patient's femur are disclosed herein. In one embodiment, a telescopic assembly for supporting a patient's femur comprises an elongate tubular base configured to be fixably attached to a table. The tubular base has a longitudinal axis. In addition, the telescopic assembly comprises an elongate extension member slidably disposed in the tubular base and configured to telescopically extend longitudinally from the tubular base. Further, the telescopic assembly comprises a femur support member coupled to an end of the extension member and configured to be positioned below and support the femur of the patient. The femur support member is configured to move longitudinally with the extension member relative to the tubular base.

Embodiments of systems for supporting a thigh and femur of a patient during a surgical or diagnostic procedure are disclosed herein. In one embodiment, a system for supporting a thigh and femur of a patient during a surgical or diagnostic procedure comprises a table having a longitudinal axis, a first end, a second end, and a pair of lateral sides extending between the first end and the second end. The table comprises a pair of laterally spaced recesses at the intersections of the lateral sides and the second end. The table also comprises a base extending from the first end to the recesses. In addition, the table comprises a horizontal extension positioned between the recesses and extending longitudinally from the base to the second end. Further, the table comprises a perineal post extending vertically from the extension at the second end of the table. Moreover, the system comprises a telescopic assembly coupled to the table along one of the lateral sides of the table. The telescopic assembly comprises an elongate tubular base fixably attached to the table. The tubular base has a longitudinal axis oriented parallel to the longitudinal axis of the table. Still further, the telescopic assembly comprises an extension member slidably disposed in the tubular base and configured to telescopically extend longitudinally from the tubular base. Still further, the telescopic assembly comprises a femur support member coupled to an end of the extension member distal the tubular base and configured to support the thigh and femur of the patient. The femur support member is configured to move longitudinally with the extension member relative to the tubular base.

Embodiments described herein comprise a combination of features and characteristics intended to address various shortcomings associated with certain prior devices, systems, and methods. The foregoing has outlined rather broadly the features and technical characteristics of the disclosed embodiments in order that the detailed description that follows may be better understood. The various characteristics and features described above, as well as others, will be readily apparent to those skilled in the art upon reading the following detailed description, and by referring to the accompanying drawings. It should be appreciated that the conception and the specific embodiments disclosed may be readily utilized as a basis for modifying or designing other structures for carrying out the same purposes as the disclosed embodiments. It should also be realized that such equivalent constructions do not depart from the spirit and scope of the principles disclosed herein.

### BRIEF DESCRIPTION OF THE DRAWINGS

For a detailed description of the preferred embodiments of the invention, reference will now be made to the accompanying drawings in which:

FIG. 1 is a top view of an embodiment of a system in accordance with the principles described herein including independently longitudinally adjustable femur support members for providing support to one or both femurs of a patient;

FIG. 2 is a bottom view of the system of FIG. 1;

FIG. 3 is a partial, enlarged top view of the system of FIG. 1 illustrating exemplary longitudinal positions of the femur support members;

FIG. 4 is a partial, enlarged side view of the system of FIG. 1 with the femur support members in the exemplary longitudinal positions shown in FIG. 3;

FIG. 5 is a partial, enlarged top view of the system of FIG. 1 illustrating additional exemplary longitudinal positions of the femur support members;

FIG. 6 is a partial, enlarged side view of the system of FIG. 1 with the femur support members in the exemplary longitudinal positions shown in FIG. 5;

FIG. 7 is a top view of the system of FIG. 1 illustrating yet additional exemplary longitudinal positions of the femur support members;

FIG. 8 is a partial, enlarged side view of an embodiment of a system in accordance with the principles described herein including independently longitudinally and vertically adjustable femur support members for providing support to one or both femurs of a patient;

FIG. 9 is a partial, enlarged side view of the system of FIG. 8 illustrating exemplary longitudinal and vertical positions of the femur support members;

FIG. 10 is a partial, enlarged side view of the system of FIG. 8 illustrating additional exemplary longitudinal and vertical positions of the femur support members;

FIG. 11 is a partial, enlarged side view of the system of FIG. 8 illustrating yet additional exemplary longitudinal and vertical positions of the femur support members;

FIG. 12 is a partial, enlarged side view of an embodiment of a system in accordance with the principles described herein including independently longitudinally and vertically adjustable femur support members for providing support to one or both femurs of a patient;

FIG. 13 is a partial, enlarged top view of an embodiment of a system in accordance with the principles described herein including independently longitudinally adjustable femur support members for providing support to one or both femurs of a patient;

FIG. 14 is a partial, enlarged top view of an embodiment of a system in accordance with the principles described herein including independently longitudinally adjustable femur support members for providing support to one or both femurs of a patient;

FIG. 15 is a top view of an embodiment of a system in accordance with the principles described herein including independently longitudinally and vertically adjustable femur support members for providing support to one or both femurs of a patient;

FIG. 16 is a partial, perspective top view of the system of FIG. 15;

FIG. 17 is a partial, perspective side view of the system of FIG. 15;

FIG. 18 is a cross-sectional, partial bottom view of one of the telescoping side rails of FIG. 15;

FIG. 19 is a cross-sectional, perspective end view of the locking mechanism of one of the telescoping side rails of FIG. 15;

FIG. 20 is an enlarged, partial, perspective bottom view of the system of FIG. 15 illustrating one femoral support and the corresponding linear vertical height adjustment assembly and rotational vertical height adjustment assembly;

FIG. 21 is a cross-sectional, perspective side view of one of the telescoping side rails and the corresponding linear vertical height adjustment assembly of FIG. 15;

FIG. 22 is a rear, perspective, partial cross-sectional view of the femoral support and the corresponding linear vertical height adjustment assembly and rotational vertical height adjustment assembly of FIG. 20;

FIG. 23 is an enlarged top view of the femoral support and the corresponding linear vertical height adjustment assembly and rotational vertical height adjustment assembly of FIG. 20;

FIGS. 24a and 24b are enlarged front and front cross-sectional views, respectively, of the locking assembly of the rotational vertical height adjustment assembly of FIG. 20 in the locked position;

FIGS. 25a and 25b are enlarged front and front cross-sectional views, respectively, of the locking assembly of the rotational vertical height adjustment assembly of FIG. 20 in the unlocked position;

FIGS. 26-28 are bottom views of an embodiment of an imaging cassette support assembly in accordance with the principles described herein used in connection with the system of FIG. 8 and illustrating the imaging cassette in several exemplary longitudinal positions;

FIG. 29 is a bottom view of an embodiment of an imaging cassette support assembly in accordance with the principles described herein used in connection with the system of FIG. 8;

FIG. 30 is a bottom view of an embodiment of an imaging cassette support assembly in accordance with the principles described herein used in connection with the system of FIG. 8;

FIG. 31 is a bottom view of an embodiment of an imaging cassette support assembly in accordance with the principles described herein used in connection with the system of FIG. 8;

FIG. 32 is a bottom view of an embodiment of an imaging cassette support assembly in accordance with the principles described herein used in connection with the system of FIG. 8;

FIG. 33 is a bottom view of an embodiment of an imaging cassette support assembly in accordance with the principles described herein used in connection with the system of FIG. 8;

FIG. 34 is a bottom view of an embodiment of an imaging cassette support assembly in accordance with the principles described herein used in connection with the system of FIG. 8;

FIG. 35 is a bottom view of an embodiment of an imaging cassette support assembly in accordance with the principles described herein used in connection with the system of FIG. 8;

FIG. 36 is a bottom view of an embodiment of an imaging cassette support assembly in accordance with the principles described herein used in connection with the system of FIG. 8; and

FIGS. 37 and 38 are bottom views of an embodiment of an imaging cassette support assembly in accordance with the principles described herein used in connection with the system of FIG. 8.

### DETAILED DESCRIPTION OF THE PREFERRED EMBODIMENTS

The following discussion is directed to various exemplary embodiments. However, one skilled in the art will understand that the examples disclosed herein have broad application, and that the discussion of any embodiment is meant only to be exemplary of that embodiment, and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct engagement between the two devices, or through an indirect connection that is established via other devices, components, nodes, and connections. In addition, as used herein, the terms “axial” and “axially” generally mean along or parallel to a particular axis (e.g., central axis of a body or a port), while the terms “radial” and “radially” generally mean perpendicular to a particular axis. For instance, an axial distance refers to a distance measured along or parallel to the axis, and a radial distance means a distance measured perpendicular to the axis. Any reference to up or down in the description and the claims is made for purposes of clarity, with “up,” “upper,” and “upwardly,” meaning vertically away from the ground or operating room (OR) floor and with “down,” “lower,” and “downwardly,” meaning vertically toward the ground or the OR floor. As used herein, the terms “approximately,” “about,” “substantially,” and the like mean within 10% (i.e., plus or minus 10%) of the recited value. Thus, for example, a recited angle of “about 80 degrees” refers to an angle ranging from 72 degrees to 88 degrees. Additionally, as used herein, the terms “bed” and “table” generally refer to a patient bed, operating table, an examination bed, a removable table top, or any other medical bed or table used for medical procedures, operations, diagnostics, and care. Further, as used herein, the term “elongate” refers to a structure or component that has a length that is substantially greater (e.g., at least twice as great) than its width, thickness, diameter, or other dimension.

Some conventional devices for supporting a patient's thigh and femur during anterior hip replacement surgery include a stationary (i.e., non-movable) thigh support pad that is fixably coupled to the operating table adjacent a stationary perineal post extending vertically upward from the operating table. Accordingly, the support pad cannot be moved longitudinally relative to the longitudinal axis of the table or vertically relative to the table. However, due to differences in anatomy of different patients and differences in sizes of different patients, the desired or optimal position of the thigh support for providing optimal exposure of the femur may vary from patient to patient. Unfortunately, with conventional operating tables for performing hip replacement surgeries having stationary perineal posts and stationary thigh support pads, it may be difficult if not impossible to support a patient's thigh and femur at the optimal orientation, which may result in additional complexities for the surgeon and/or less than optimal surgical results.

Some other conventional devices for supporting a patient's thigh and femur during surgeries may allow for longitudinal adjustment of the femur support pad and/or vertical adjustment of the femur support pad, but may not allow for (a) independent adjustment of the longitudinal position and vertical position of the femur support pad, (b) incremental adjustment of the longitudinal position and/or vertical position of the femur support pad, or (c) a sufficient range of motion in the longitudinal and/or vertical directions. These limitations inhibit or eliminate the ability of the surgeon to isolate movement of the femur support pad to purely longitudinal movements (without any vertical component of movement) or purely vertical movements (without any longitudinal movements), and/or to easily controllably limit the longitudinal adjustments and vertical adjustments, thereby making it difficult for the surgeon to “dial in” the desired or optimal position of the femur support pad during a procedure. Still further, many conventional devices for supporting a patient's thigh and femur during surgeries include structural features (e.g., spars) positioned below the table and between the patient's legs, which interfere with and prevent positioning of an X-ray cassette below the table and patient's leg(s).

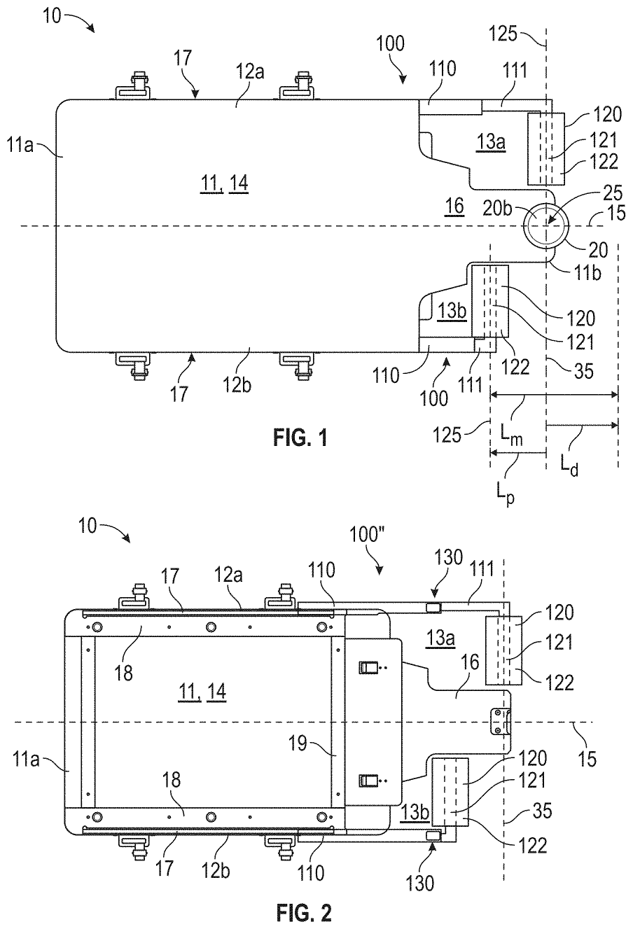

Referring now to FIGS. 1-7, an embodiment of a system 10 for directly supporting a patient's thighs, thereby supporting and positioning the patient's femurs as desired during a surgical or diagnostic procedure is shown. In this embodiment, system 10 can be used to independently support and position one or both of the patient's femurs. For example, system 10 can be used to independently support and position the patient's femur undergoing or otherwise involved in the procedure, referred to herein as the “operative” femur or side of the patient, and also independently support and position the patient's opposite femur (i.e., the patient's femur that is not the subject of the procedure), referred to herein as the “non-operative” femur or side. Although systems disclosed herein can be modified to support only the operative femur of the patient in some embodiments, support of both femurs offers the potential to reduce undesirable pelvic tilt or rotation of the patient, particularly when positioning the patient on system 10.

In this embodiment, system 10 includes a base 11 and a pair of telescopic femur support assemblies 100 removably secured to base 11. In this embodiment, base 11 is a flat, generally board-like structure configured to support a patient, and in particular, the sacral region of a patient. In some embodiments, base 11 is an operating table or bed (or part thereof), whereas in other embodiments, base 11 is a structure that is removably coupled to an operating table or bed. It should be appreciated that base 11 may include a resilient pad to enhance the comfort of a patient laying on base 11. In any of the foregoing embodiments, system 10 is fully and completely supported by the operating table or bed, and thus, does not require an additional support or stand. Base 11 is horizontally oriented, and thus, has a horizontal central or longitudinal axis 15, a first end 11a, a second end 11b axially opposite end 11a, and lateral sides 12a, 12b extending between ends 11a, 11b. In addition, base 11 includes recesses 13a, 13b at the intersections of end 11b and lateral sides 11a, 11b, respectively. As a result, base 11 may be described as including a generally rectangular portion 14 extending axially from end 11a to recesses 13a, 13b and an extension 16 projecting axially from portion 14 to end 11b. Extension 16 is positioned between recesses 13a, 13b and is laterally centered between sides 12a, 12b. A perineal post 20 is fixably mounted to extension 16 at end 11b. Perineal post 20 extends vertically upward from extension 16 and is laterally centered relative to sides 11a, 11b. Consequently, perineal post 20 has a vertically oriented central axis 25, a first or lower end 20a fixably secured to extension 16 at end 11b, and a second or upper end 20b distal extension 16. Central axis 25 intersects and is oriented perpendicular to longitudinal axis 15. A horizontally oriented reference axis 35 is orthogonal to both longitudinal axis 15 and central axis 25 of perineal post 20. An annular pad may be provided on perineal post 20. Similar to base 11, perineal post 20 may include a resilient pad to enhance patient comfort.

As best shown in FIGS. 2 and 4, a pair of elongate rigid rails 17 are fixably coupled to the bottom of portion 14 and extend longitudinally (relative to axis 15) along lateral sides 12a, 12b. Additional structural support members for supporting the weight of the patient may be mounted to the bottom of base 11 such as elongate, rigid beams 18 extending longitudinally along lateral sides 12a, 12b from end 11a to recesses 13a, 13b and elongate, rigid cross-members 19 extending laterally between beams 18. In this embodiment, rails 17 are fixably attached to beams 18, which in turn are fixably attached to portion 14 of base 11.

Referring now to FIGS. 1 and 4, in this embodiment, telescopic femur support assemblies 100 are removably secured to rails 17. One telescopic femur support assembly 100 will now be described it being understood that both telescopic femur support assemblies 100 are the same. In this embodiment, telescopic femur support assembly 100 includes an elongate, rigid tubular base 110 fixably and removably coupled to rail 17 of table 11, an elongate, rigid extension member 111 slidingly and telescopically received in tubular base 110, and a femur support member 120 fixably coupled to the end of extension member 111 distal base 110. When tubular base 110 is fixably attached to rail 17 and table 11, tubular base 110 cannot move translationally or rotationally relative to table 11.

As best shown in FIG. 4, in this embodiment, a rigid vertical post 112 is fixably attached to the distal end of extension member 111 and femur support member 120 is fixably attached to the upper end of post 112. Tubular base 110 and extension member 111 are coaxially aligned, each sharing a common central or longitudinal axis oriented parallel to longitudinal axis 15. Although telescopic femur support assemblies 100 are removably secured to rails 17 in this embodiment, in other embodiments, the telescopic femoral support assemblies 100 may be removably secured to or integrated with beams 18. It should be appreciated that telescopic femur support assemblies 100 are coupled to and cantilevered from rails 17, and thus, loads applied to the telescopic femur support assemblies 100 (e.g., the load of a patient's leg) are transferred through assemblies 100 to rails 17. Consequently, rails 17, as opposed to portion 14 or extension 16, supports loads applied to telescopic femur support assemblies 100.

As best shown in FIG. 1, femur support member 120 has a generally cylindrical geometry and is horizontally oriented. More specifically, femur support member 120 includes a elongate, rigid post 121 and an elongate annular pad 122 disposed about and mounted to post 121. Post 121 has a horizontally oriented central axis 125, a laterally outer “fixed” end that is fixably attached to the upper end of vertical post 112, and a laterally inner “free” end medial of vertical post 112. In this embodiment, pad 122 is coaxially aligned with post 121 such that they share common central axis 125, which is oriented perpendicular to axis 15 in top view. It should be appreciated that pads 122 are positioned immediately below and directly support the patient's legs when the patient is positioned on the base 11 with the patient's head proximal end 11a, the patient's legs on opposite sides of perineal post 20, and the patient's pelvis positioned against perineal post 20.

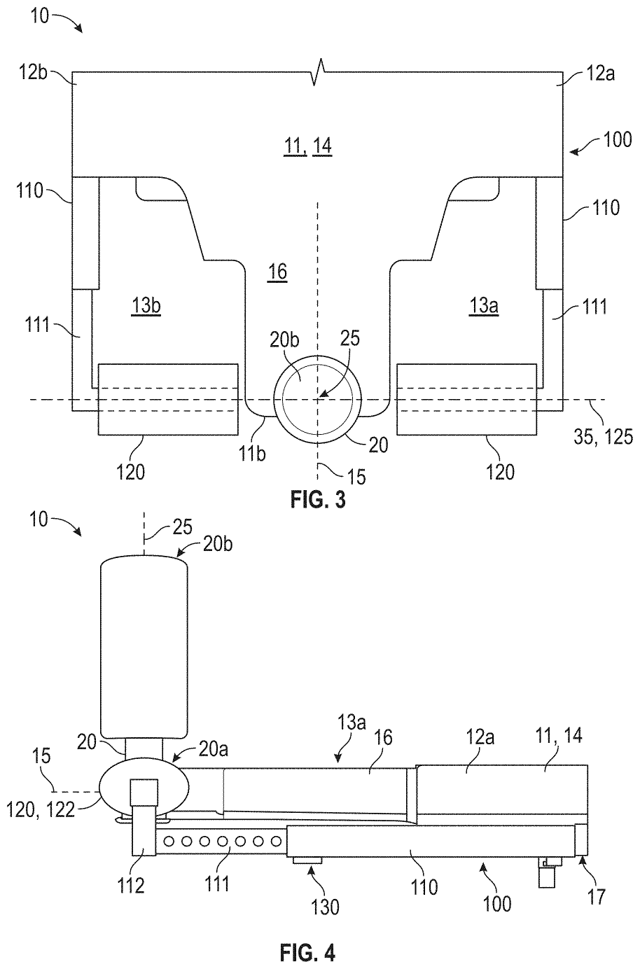

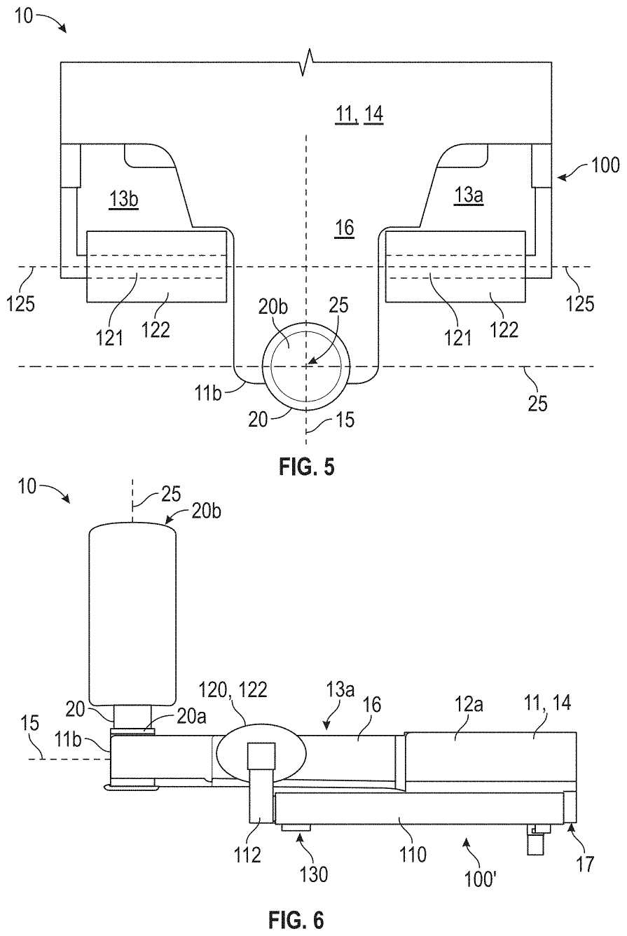

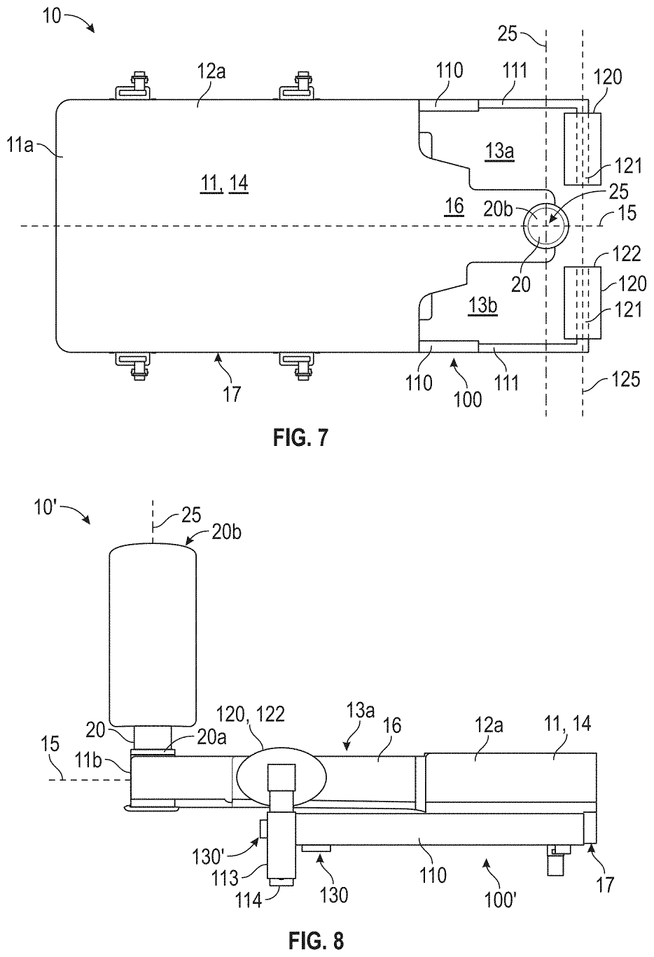

Referring now to FIG. 4, tubular base 110 and extension member 111 are coaxially aligned and oriented parallel to longitudinal axis 15. Thus, tubular base 110 and extension member 111 are horizontally oriented and share a common central or longitudinal axis oriented parallel to axis 15. Extension member 111 is slidingly disposed in base 110 such that it can move longitudinally (i.e., parallel to axis 15) relative to base 110 and extend longitudinally and telescopically therefrom, but cannot rotate or move vertically relative to base 110. In this embodiment, extension member 111 and base 110 have similar cross-sectional geometries (e.g., both have rectangular cross-sectional geometries) such that the outer surface of extension member 111 generally mates with and slidingly engages the inner surface of base 110. Longitudinal movement of extension member 111 relative to base 110 is used to position femur support member 120 at the desired longitudinal position relative to the patient and perineal post 20. As shown in FIGS. 1-6, femur support members 120 of telescopic femur support assemblies 100 can be moved independently and longitudinally relative to base 11 and perineal post 20 to position femur support members 120 at the desired positions relative to the patient. For example, in FIGS. 1 and 2, femur support members 120 are disposed at different longitudinal positions; in FIGS. 3 and 4, femur support members 120 are longitudinally aligned with each other and perineal post 20; in FIGS. 5 and 6, femur support members 120 are aligned with each other and longitudinally positioned within recesses 13a, 13b between perineal post 20 and portion 14; and in FIG. 7, femur support members 120 are aligned with each other and longitudinally positioned outside of recesses 13a, 13b and distal perineal post 20, portion 14, and extension 16.

Referring now to FIG. 4, each telescopic femur support assembly 100 can be extended and contracted by moving extension member 111 longitudinally relative to the corresponding base 110. It should be appreciated that longitudinal movement of extension member 111 relative to base 110 results in longitudinal movement of femur support member 120 coupled to distal end of extension member 111, and further, such longitudinal movement of extension member 111 and femur support member 120 is purely longitudinal meaning there is no vertical or lateral component associated with the longitudinal movement of extension member 111 and femur support member 120. In addition, each telescopic femur support assembly 100 includes a locking mechanism 130 that can be actuated to lock the relative longitudinal positions of base 110 and extension member 111 (i.e., prevent extension member 111 from moving longitudinally relative to base 110), thereby locking the longitudinal position of the corresponding femur support member 120 relative to perineal post 20 and base 11. Thus, when extension member 111 is locked relative to base 110, extension member 111 cannot be moved longitudinally relative to base 110, however, when extension member 111 is unlocked from base 110, extension member 111 can be moved longitudinally relative to base 110. In general, locking mechanism 130 can be any device for selectively and controllably locking and unlocking extension member 111 to base 110 to prevent and allow, respectively, extension member 111 to move longitudinally relative to base 110. In this embodiment, locking mechanism 130 is a manual push button extending from base 110 that can be selectively actuated by a user (e.g., surgeon) to move a detent out of engagement with one of a plurality of longitudinally spaced mating holes in extension member 111. The detent is biased into engagement with the holes (i.e., biased into the locked position), which are uniformly longitudinally spaced along extension member 111 to allow the user to longitudinally slide extension member 111 relative to base 110 between a plurality of predetermined intervals. Thus, locking mechanism 130 enables controlled, uniform, incremental longitudinal movement of extension member 111 relative to base 110, and hence, the controlled, uniform, incremental longitudinal movement and positioning of femur support member 120 coupled to the distal end of extension member 111.

As described above, in this embodiment, extension member 111 can be manually moved relative to base 110 and manually locked/unlocked relative to base 110. However, in other embodiments, the extension member (e.g., extension member 111) can be electronically actuated to move longitudinally relative to the base (e.g., base 110) and/or the locking mechanism can be electronically actuated to lock and unlock the extension member relative to the base.

With the patient disposed on base 11 as described above, femur support members 120 can be moved longitudinally along the bottom of the patient's thigh to support the corresponding femurs of the patient at the desired and/or optimal longitudinal positions. It should be appreciated that the ability to independently and controllably adjust the longitudinal position of each femur support member 120 enables positioning of each femur support member 120 at the desired and/or optimal longitudinal positions regardless of the size of the patient or potential variations in the anatomy of any given patient. For example, for certain hip surgeries, it may be particularly desirable to longitudinally position a femur support member 120 such that axis 125 is aligned with and positioned below the lesser trochanter of the affected leg in top view. As best shown in FIG. 1, each telescopic femur support assembly 100 is configured such that the corresponding extension member 111 and femur support member 120 has a longitudinal range of motion Lₘ measured longitudinally relative to perineal post 20 and reference axis 35. More specifically, the longitudinal range of motion Lₘ of each extension member 111 and corresponding femur support member 120 includes a proximal range of motion Lₚ measured longitudinally and proximally from perineal post 20 and reference axis 35 to central axis 125 of the femur support member 120, and a distal range of motion L_{d} measured longitudinally and distally from perineal post 20 and reference axis 35 to central axis 125 of the femur support member 120. In this embodiment, central axis 125 coincides with the central axis of the corresponding annular pad 122, and thus, the proximal range of motion Lₚ and the distal range of motion L_{d} can also be measured longitudinally from perineal post 20 and reference axis 35 to the central axis of pad 122. To accommodate most patients of different sizes and anatomies, in embodiments described herein, each telescopic femur support assembly (e.g., telescopic femur support assembly 100) is sized and arranged such that the longitudinal range of motion Lₘ of each femur support member (e.g., femur support member 120) ranges from 100 mm to 165 mm; the proximal range of motion Lₚ ranges from 0 mm to 120 mm, alternatively ranges from 0 to 110 mm, and alternatively ranges from 0 mm to 100 mm; and the distal range of motion L_{d} ranges from 0 mm to 45 mm, alternatively ranges from 0 mm to 20 mm, and alternatively is 0 mm. Stated differently, and with longitudinal distances measured proximal to perineal post 20 and reference axis 35 (i.e., distances measured from perineal post 20 and reference axis 35 toward portion 14 as denoted by proximal range of motion Lₚ) being expressed as positive distances and longitudinal distances measured distal to perineal post 20 and reference axis 35 (i.e., distances measured from perineal post 20 and reference axis 35 away from portion 14 as denoted by distal range of motion L_{d}) being expressed in the negative, in embodiments described herein, the longitudinal range of motion Lₘ of each femur support member ranges from −45 mm to 120 mm, alternatively ranges from −20 mm to 110 mm, and alternatively ranges from 0 mm to 100 mm.

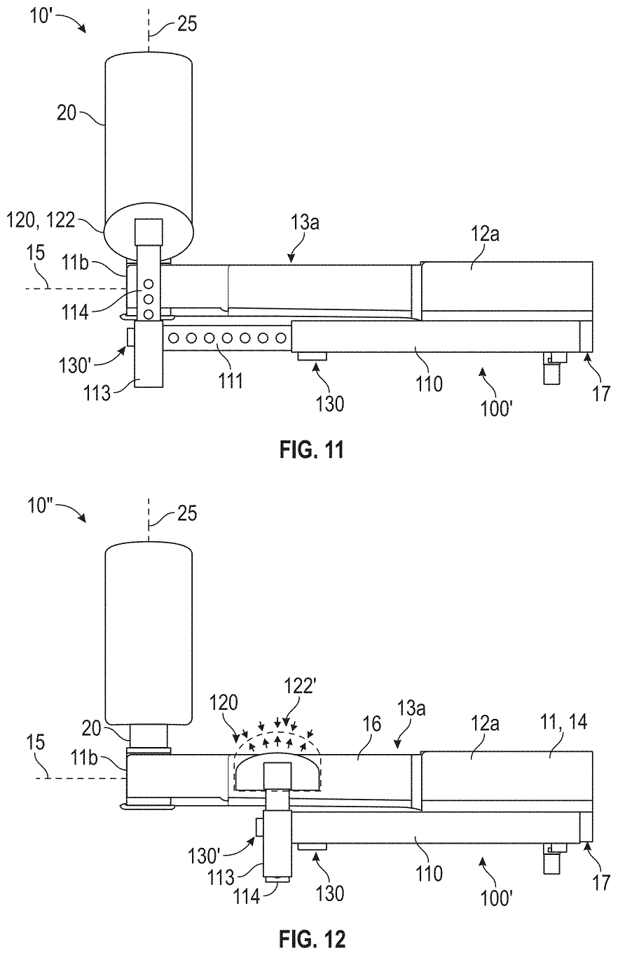

Referring now to FIGS. 8-11, another embodiment of a system 10′ for directly supporting a patient's thighs, thereby supporting and positioning the patient's femurs as desired during a surgical or diagnostic procedure is shown. System 10′ is substantially the same as system 10 previously described, with the exception that the femur support members 120 can also be selectively and controllably, vertically adjusted independent of any longitudinal adjustment. Accordingly, the differences between systems 10, 10′ will be described it being understood the other features are generally the same. In addition, for purposes of clarity, like components between systems 10, 10′ are given the same reference numerals.

As shown in FIGS. 8-11, in this embodiment, system 10′ includes an operating table or base 11 and a pair of telescopic femur support assemblies 100′ removably secured to base 11. Base 11 is as previously described and shown in FIG. 1. Telescopic femur support assemblies 100′ are similar to telescopic femur support assemblies 100 previous described and shown in FIG. 1. In particular, telescopic femur support assemblies 100′ are removably secured to rails 17 of base 11. In addition, each telescopic femur support assembly 100′ includes an elongate tubular base 110 fixably and removably coupled to rail 17, an extension member 111 slidingly and telescopically received by tubular base 110, and a femur support member 120 coupled to the end of extension member 111 distal base 110. Thus, longitudinal movement of extension member 111 and femur support member 120 is purely longitudinal (i.e., there is no vertical or lateral component associated with the longitudinal movement of extension member 111 and femur support member 120), and further, locking mechanism 130 enables extension member 111 and femur support member 120 to be moved longitudinally in a controlled, uniform, incremental manner. However, unlike support assemblies 100 previously described, in this embodiment, femur support members 120 are not fixably attached to the end of extension member 111. Rather, in this embodiment, femur support members 120 are moveably coupled to the ends of the corresponding extension members 111 such that femur support members 120 can be independently, selectively, and controllably moved in the vertical direction relative to the corresponding extension members 111 and base 11. Specifically, each telescopic femur support assembly 100′ includes a rigid, tubular sleeve 113 fixably attached to the distal end of extension member 111 and a rigid extension post 114 slidingly and telescopically received by tubular sleeve 113. Femur support member 120 is fixably attached to the upper end of extension post 114. Although telescopic femur support assemblies 100′ are removably secured to rails 17 in this embodiment, in other embodiments, the telescopic femoral support assemblies 100′ may be removably secured to or integrated with beams 18.

Tubular sleeve 113 and extension post 114 are coaxially aligned and vertically oriented (i.e., oriented parallel to the central axis of perineal post 20). In addition, extension post 114 is slidingly disposed in sleeve 113 such that it can move vertically relative to sleeve 113 and extend vertically therefrom, but cannot rotate or move horizontally relative to sleeve 113. In this embodiment, extension post 114 and sleeve 113 have similar cross-sectional geometries (e.g., both have rectangular cross-sectional geometries) such that the outer surface of extension post 114 generally mates with and slidingly engages the inner surface of sleeve 113. Vertical movement of extension post 114 relative to sleeve 113 is used to position femur support member 120 at the desired vertical position relative to the patient and table 11. Thus, in this embodiment, the positions of femur support members 120 of telescopic femur support assemblies 100′ can be independently, selectively, and controllably adjusted in both the longitudinal and vertical directions.

Each extension post 114 can be extended and contracted by moving extension post 114 vertically relative to the corresponding sleeve 113. It should be appreciated that vertical movement of extension post 114 relative to sleeve 113 results in vertical movement of femur support member 120 coupled to the upper end of extension post 114, and further, such vertical movement of extension post 114 and femur support member 120 is purely vertical meaning there is no longitudinal or lateral component associated with the vertical movement of extension post 114 and femur support member 120. Similar to locking mechanism 130 for locking the longitudinal position of the corresponding femur support member 120 relative to perineal post 20 and base 11, each telescopic femur support assemblies 100′ includes a locking mechanism 130′ to lock the vertical position of the corresponding femur support member 120 relative to the corresponding sleeve 113, extension member 111, and base 110. Thus, when extension post 114 is locked relative to sleeve 113, extension post 114 cannot be moved vertically relative to sleeve 113, however, when extension post 114 is unlocked from sleeve 113, extension post 114 can be moved vertically relative to sleeve 113. In general, locking mechanism 130′ can be any device for selectively and controllably locking and unlocking extension post 114 to sleeve 113 to prevent and allow, respectively, extension post 114 to move vertically relative to sleeve 113. Similar to locking mechanism 130, in this embodiment, locking mechanism 130′ is a manual push button extending from sleeve 113 that can be selectively actuated by a user (e.g., surgeon) to move a detent out of engagement with one of a plurality of vertically-spaced mating holes in extension post 114. The detent is biased into engagement with the holes (i.e., biased into the locked positon), which are uniformly vertically spaced along extension post 114 to allow the user to vertically slide extension post 114 relative to sleeve 113 between a plurality of predetermined intervals. Thus, locking mechanism 130′ enables controlled, uniform, incremental vertical movement of extension post 114 relative to sleeve 113, and hence, the controlled, uniform, incremental vertical movement and positioning of femur support member 120 relative to sleeve 113, extension member 111, and base 110.

With the patient disposed on base 11 as described above, femur support members 120 of telescopic femur support assemblies 100′ can be moved longitudinally and vertically to support the corresponding femurs of the patient at the desired and/or optimal positions. It should be appreciated that the ability to independently, selectively, and controllably adjust the longitudinal and vertical positions of each femur support member 120 of each telescopic femur support assembly 100′ (independent of each other) enables positioning of each femur support member 120 at the desired and/or optimal positions regardless of the size of the patient or potential variations in the anatomy of any given patient.

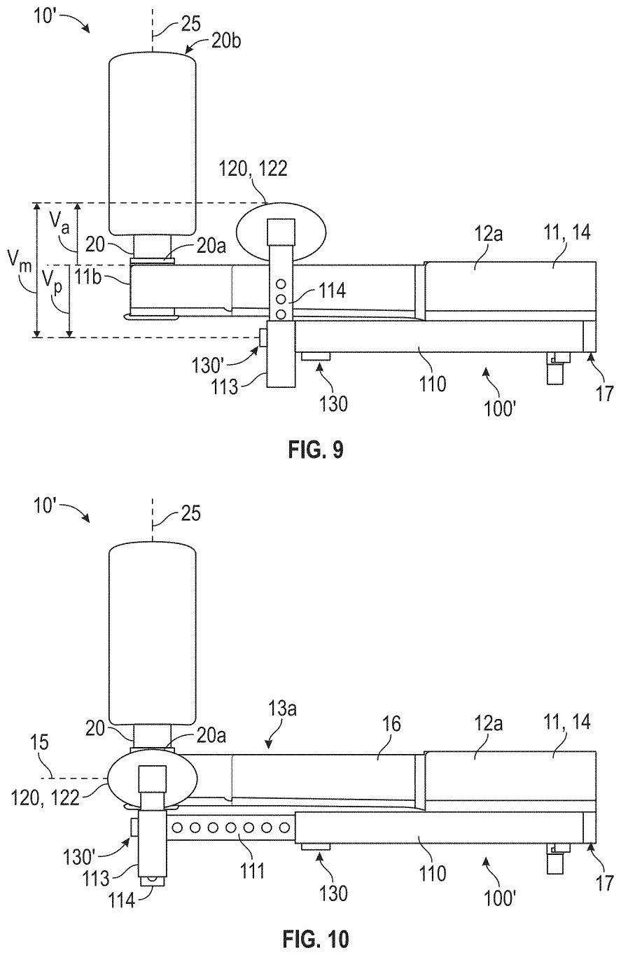

As best shown in FIG. 9, each telescopic femur support assembly 100′ is configured such that the corresponding femur support member 120 has a vertical range of motion Vₘ measured vertically and parallel to perineal post 20 and central axis 25. More specifically, the vertical range of motion Vₘ of each femur support member 120 includes an anterior range of motion Vₐ measured vertically upward and anteriorly from a horizontal plane containing the horizontal upper surface of base 11 and extension 16 to a parallel horizontal plane passing through the uppermost, anterior peak or crest of the annular pad 122 when extension post 114 is fully extended vertically upward relative to sleeve 113; and a posterior range of motion Vₚ measured vertically downward and posterior from the horizontal plane containing the horizontal upper surface of base 11 and extension 16 to the parallel horizontal plane passing through the uppermost, anterior peak or crest of the annular pad 122 when extension post 114 is fully retracted vertically downward relative to sleeve 113. To accommodate most patients of different sizes and anatomies, in embodiments described herein, each telescopic femur support assembly (e.g., telescopic femur support assembly 100′) is sized and arranged such that the vertical range of motion Vₘ of each femur support member (e.g., femur support member 120) ranges from 40 mm to 130 mm; the anterior range of motion Vₐ ranges from 0 mm to 80 mm, alternatively ranges from 0 mm to 60 mm, and alternatively ranges from 0 mm to 40 mm; and the posterior range of motion Vₚ ranges from 0 mm to 50 mm, alternatively ranges from 0 mm to 25 mm, and alternatively is 0 mm. Stated differently, and with vertical distances measured upward and anteriorly to the upper surface of extension 16 and base 11 (i.e., as denoted by anterior range of motion Vₐ) being expressed as positive distances and vertical distances measured downward and posteriorly to the upper surface of extension 16 and base 11 (i.e., as denoted by posterior range of motion Vₚ) being expressed in the negative, in embodiments described herein, the vertical range of motion Vₘ of each femur support member ranges from −50 mm to 80 mm, alternatively ranges from −25 mm to 60 mm, and alternatively ranges from 0 mm to 40 mm.

As described above, in this embodiment, extension member 111 can be manually moved relative to base 110 and manually locked/unlocked relative to base 110, and post 114 can be manually moved relative to sleeve 113 and manually locked/unlocked relative to sleeve 113. However, in other embodiments, the extension member (e.g., extension member 111) can be electronically actuated to move longitudinally relative to the base (e.g., base 110) and/or the locking mechanism can be electronically actuated to lock and unlock the extension member relative to the base; and the post (e.g., post 114) can be electronically actuated to move vertically relative to the sleeve (e.g., sleeve 113) and/or the locking mechanism can be electronically actuated to lock and unlock the post relative to the sleeve.

Referring now to FIG. 12, another embodiment of a system 10″ for directly supporting a patient's thighs, thereby supporting and positioning the patient's femurs as desired during a surgical or diagnostic procedure is shown. System 10″ is substantially the same as system 10′ previously described, with the exception that the femur support members 120 are replaced with expandable femur support members 120′. In this embodiment, each femur support member 120′ has a generally semi-cylindrical geometry and is horizontally oriented. In addition, each expandable femur support member 120′ is fixably coupled to the upper end of extension post 114. More specifically, each femur support member 120′ includes an elongate post 121 as previously described and an elongate expandable pad 122′ mounted to post 121. Pad 122′ can be selectively and controllably expanded to move radially outward and vertically upward relative to central axis 125, and selectively and controllably contracted to move radially inward and vertically downward relative to central axis 125 as schematically illustrated by the arrows in FIG. 12. Controllable expansion and contraction of pad 122′ provides an additional and alternative technique for adjusting the vertical position of the patient's thigh and femur. In this embodiment, pads 122′ can be independently, selectively, and controllably expanded and contracted by a user via inflation and deflation, respectively, by pumping air into and from pads 122′, respectively.

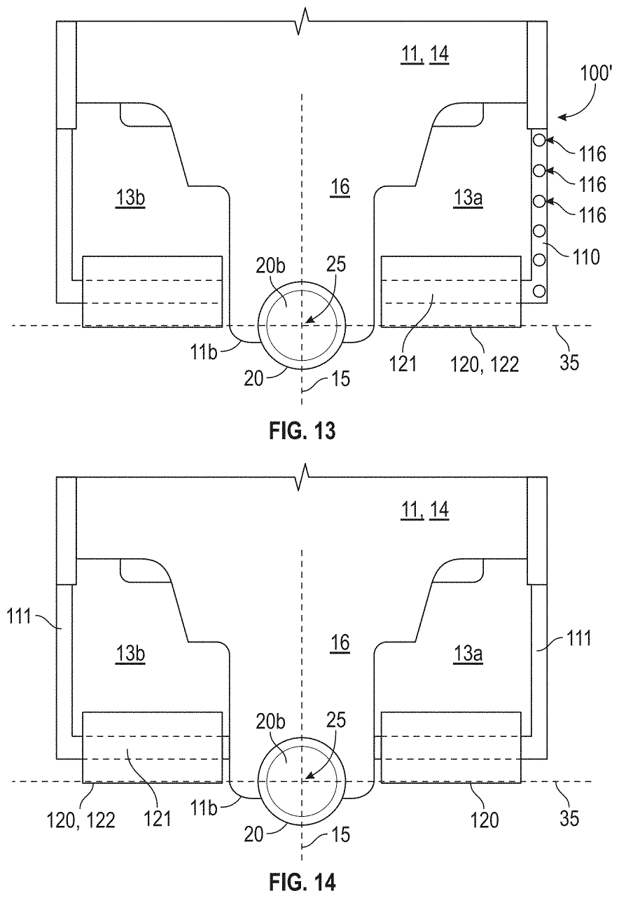

Referring briefly to FIG. 13, in some embodiments, one or both extension members 111 may include a plurality of horizontally spaced, holes or recesses 116 extending vertically downward from the upper surface of extension member(s) 111. Such holes or recesses 116 can be used to releasably mount one or more sterile devices (e.g., a sterile femoral hook) at a desired longitudinal position along the corresponding extension member 111.

In the embodiment of telescopic femur support assemblies 100 previously described, post 121 of femur support member 120 has a laterally outer “fixed” end fixably attached to the upper end of vertical post 112 and a laterally inner “free” end medial of vertical post 112. However, as shown in FIG. 14, to enhance the support strength of post 121 and femur support member 120, in some embodiments, the laterally inner end of one or both posts 121 of femur support members 120 may slidably engage and be movably supported by extension 16. For example, in FIG. 14, the laterally inner end of post 121 is slidably disposed in and slidably engages a mating, horizontally oriented, elongate recess extending horizontally into the laterally outer side of extension 16. However, it should be appreciated that in such embodiments, femur support members 120 can move longitudinally relative to extension 16 and perineal post 20, but may not be able to move vertically up or down relative thereto.

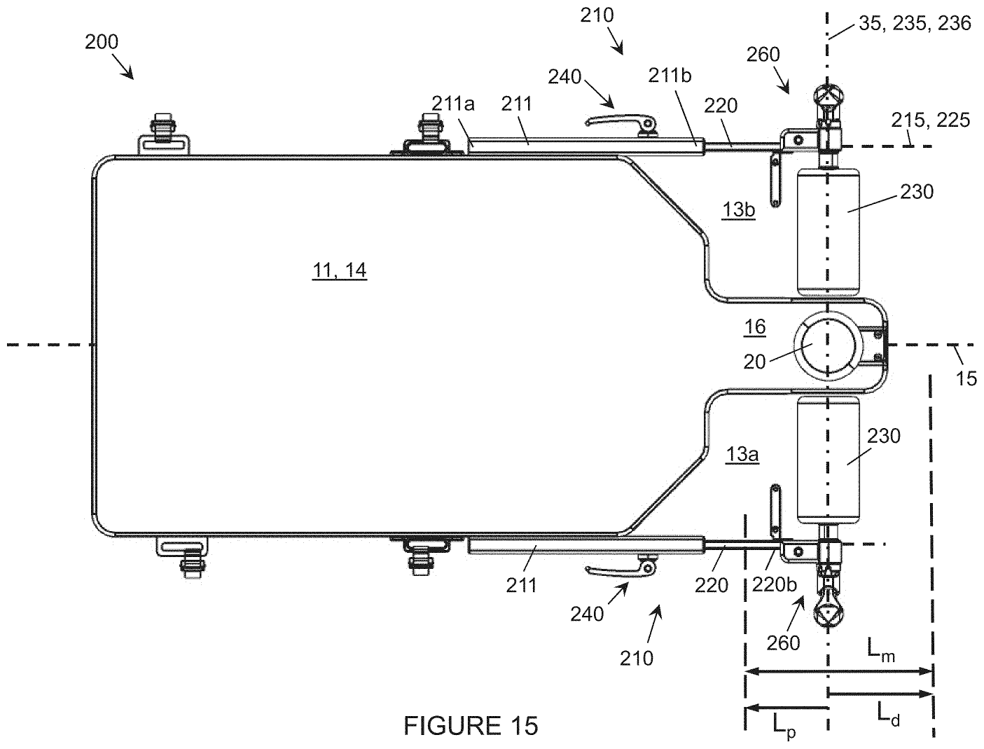

Referring now to FIGS. 15-17, an embodiment of a system 200 for directly supporting a patient's thighs, thereby supporting and positioning the patient's femurs as desired during a surgical or diagnostic procedure is shown. In this embodiment, system 200 can be used to independently support and position one or both of the patient's femurs. For example, system 200 can be used to independently support and position the patient's operative femur and/or non-operative femur. As previously described, although systems disclosed herein can be modified to support only the operative femur of the patient in some embodiments, support of both femurs offers the potential to reduce undesirable pelvic tilt or rotation of the patient, particularly when positioning the patient on system 200.

System 200 is similar to system 10′ previously described. Accordingly, the differences between systems 10′, 200 will be described it being understood the other features are generally the same. In addition, for purposes of clarity, like components between systems 10′, 200 are given the same reference numerals. In this embodiment, system 200 includes a base 11 as previously described, a perineal post 20 as previously described mounted to extension 16, and a pair of telescopic femur support assemblies 210 removably and fixably secured to base 11. More specifically, telescopic femur support assemblies 210 are removably and fixably secured to beams 18. Although telescopic femur support assemblies 210 are removably secured to beams 18 in this embodiment, in other embodiments, the telescopic femoral support assemblies 210 may be removably secured to or integrated with rails 17 or other part of base 11. It should be appreciated that telescopic femur support assemblies 210 are coupled to and cantilevered from beams 18, and thus, loads applied to the telescopic femur support assemblies 210 (e.g., the load of a patient's leg) are transferred through assemblies 210 to beams 18. Consequently, beams 18, as opposed to portion 14 or extension 16, supports loads applied to telescopic femur support assemblies 210.

One telescopic femur support assembly 210 will now be described it being understood that both telescopic femur support assemblies 210 are the same. In this embodiment, telescopic femur support assembly 210 includes an elongate, rigid tubular base 211 fixably and removably coupled to beam 18 of table 11, an elongate extension member 220 slidingly and telescopically received in tubular base 211, and a femur support member 230 moveably coupled to the end of extension member 220 distal base 211. When tubular base 211 is fixably attached to table 11, tubular base 211 cannot move translationally or rotationally relative to table 11. As will be described in more detail below, femur support member 230 is movably coupled to the end of extension member 220 by a vertical adjustment assembly 260.

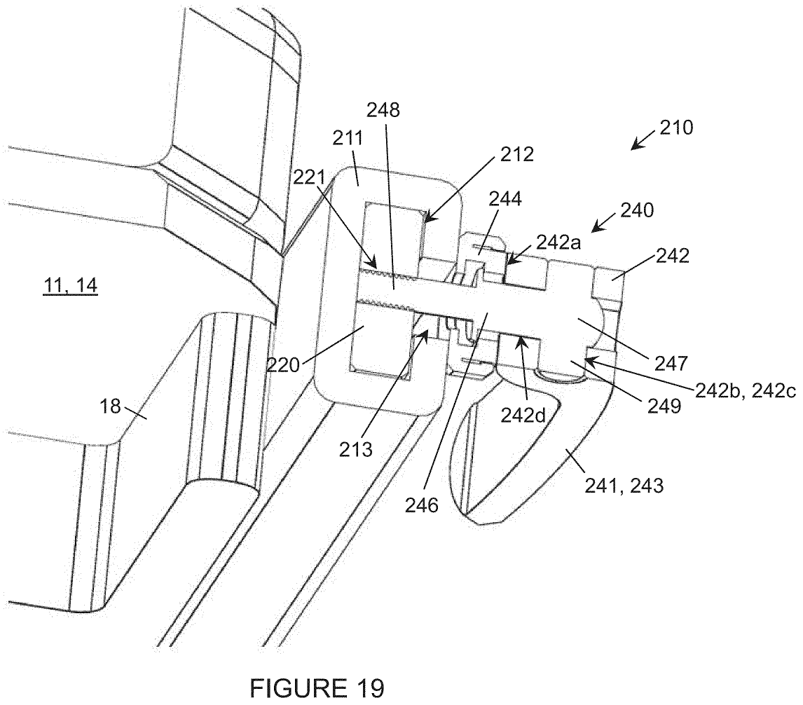

As best shown in FIGS. 15-17, tubular base 211 and extension member 220 are coaxially aligned and oriented parallel to longitudinal axis 15 of base 11. Thus, tubular base 211 and extension member 220 are horizontally oriented. Tubular base 211 has a central or longitudinal axis 215 oriented parallel to axis 15, a first or proximal end 211a fixably coupled to portion 14, a second or distal end 211b axially opposite end 211a and distal portion 14 of base 11, and a central through passage 212 extending axially from end 211a to end 211b. In this embodiment, tubular base 211 has a C-shaped cross-section in a plane oriented perpendicular to axis 215 (e.g., FIG. 19). Accordingly, tubular base 211 also includes an elongate, lateral slot 213 extending axially from end 211a to end 211b and extending radially from the outer surface of tubular base 211 to passage 212. Tubular base 211 is positioned and oriented such that slot 213 faces laterally outward and away from base 11 and central axis 15.

Extension member 220 has a central or longitudinal axis 225 coaxially aligned with axis 215 and oriented parallel to axis 15, a first or proximal end 220a disposed in passage 212 of base 211, and a second or distal end 220b axially opposite end 220a and base 11. Extension member 220 is slidingly disposed in passage 212 of base 211 and such that it can move longitudinally (i.e., parallel to axis 15) relative to base 211 and extend longitudinally and telescopically from distal end 211b of base 211, but cannot rotate or move vertically relative to base 211. In this embodiment, extension member 220 and base 211 have similar cross-sectional geometries (e.g., both have rectangular cross-sectional geometries) such that the outer surface of extension member 220 generally mates with and slidingly engages the inner surface of base 211. Longitudinal movement of extension member 220 relative to base 211 is used to position femur support member 230 at the desired longitudinal position relative to the patient and perineal post 20. Femur support members 230 of telescopic femur support assemblies 210 can be moved independently and longitudinally relative to base 11 and perineal post 20 to position femur support members 230 at the desired positions relative to the patient. In addition, each femur support member 230 can be moved longitudinally independent and separate of any vertical movement of either femur support member 230 via vertical adjustment assembly 260.

Referring again to FIGS. 15-17, each telescopic femur support assembly 210 can be extended and contracted by moving extension member 220 longitudinally relative to the corresponding base 211. It should be appreciated that longitudinal movement of extension member 220 relative to base 211 results in longitudinal movement of femur support member 230 coupled to distal end of extension member 220, and further, such longitudinal movement of extension member 220 and femur support member 230 is purely longitudinal meaning there is no vertical or lateral component associated with the longitudinal movement of extension member 220 and femur support member 230. In addition, each telescopic femur support assembly 210 includes a locking mechanism 240 that can be actuated to lock the relative longitudinal positions of base 211 and extension member 220 (i.e., prevent extension member 220 from moving longitudinally relative to base 211), thereby locking the longitudinal position of the corresponding femur support member 230 relative to perineal post 20 and base 11. Thus, when extension member 220 is locked relative to base 211, extension member 220 cannot be moved longitudinally relative to base 211, however, when extension member 220 is unlocked from base 211, extension member 220 can be moved longitudinally relative to base 211.

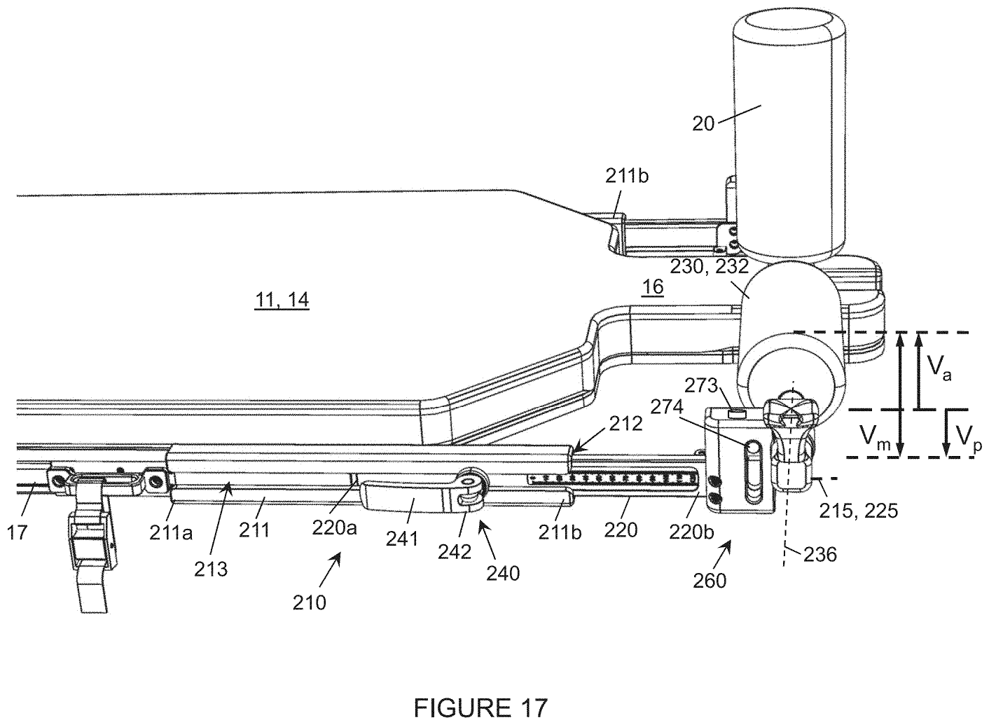

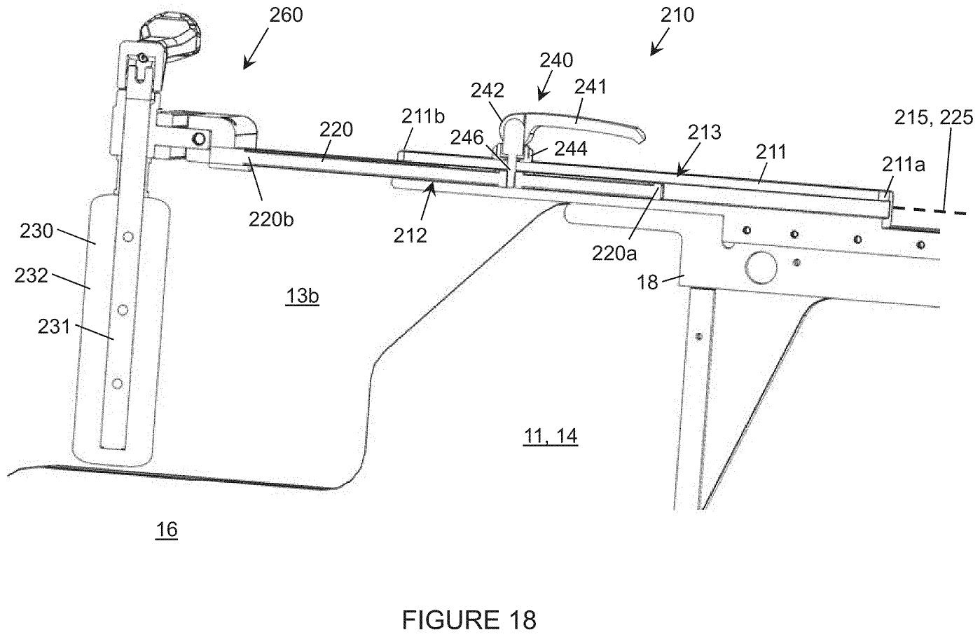

Referring now to FIGS. 17-19, in this embodiment, locking mechanism 240 is a manually actuated camming device including an actuator 241, a bearing block 244, and an elongate load transfer member 246 extending from actuator 241 through bearing block 244 and slot 213 into extension member 220. As best shown in FIG. 19, in this embodiment, load transfer member 246 is a bolt having a head 247 and an elongate shaft 248 extending from head 247. The end of shaft 248 opposite head 247 is fixably secured to extension member 220. In particular, shaft 248 is oriented perpendicular to extension member 220 and shaft 248 threadably engages a mating hole 221 in extension member 220. In other words, the end of shaft 248 opposite head 247 includes external threads that threadably engage mating internal threads in hole 221 extending through extension member 220. Head 247 of load transfer member 246 includes a cylindrical pin 249 fixably attached to shaft 248 and oriented perpendicular to shaft 248. Accordingly, load transfer member 246 is generally T-shaped.

Referring still to FIG. 19, actuator 241 includes a barrel 242 rotatably mounted to pin 249 and a handle 243 extending from barrel 242. Bearing block 244 is disposed about shaft 248 and positioned between barrel 242 and base 211. In particular, block 244 slidingly engages barrel 242 and base 211 on both sides of slot 213. Barrel 242 is a cylindrical member having a cylindrical radially outer surface 242a, a throughbore 242b defined by a radially inner cylindrical surface 242c, and a circumferentially extending slot 242d extending radially from outer surface 242a to throughbore 242b. Cylindrical outer surface 242a slidingly engages block 244, pin 249 is disposed in throughbore 242b such that cylindrical inner surface 242c slidingly engages pin 249, and shaft 248 extends through slot 242d. Barrel 242 is rotated about pin 249, while shaft 248 moves through circumferential slot 242d, with handle 243. Cylindrical outer surface 242a is not coaxially aligned with cylindrical inner surface 242c. In particular, cylindrical outer surface 242a and cylindrical inner surface 242c have central axes (i.e., each is disposed about its own central axis) that are oriented parallel to each other but are radially spaced apart. As a result, rotation of barrel 242 about pin 249 with handle 243 allows barrel 242 to function as a cam to increase and decrease compression of block 244 between base 211 and barrel 242. Namely, rotation of barrel 242 (via handle 243) about pin 249 in a first direction increases compression of block 244 between barrel 242 and base 211, and rotation of barrel 242 in a second direction opposite the first direction decreases the compression of block 244 between barrel 242 and base 211. The components of locking mechanism 240 (e.g., barrel 242, bearing block 244, and load transfer member 246) are sized and arranged such that (a) rotation of handle 243 and barrel 242 in the first direction to a “locked position” sufficiently increases compression of block 244 between base 211 and barrel 242 to effectively prevent block 244, actuator 241, load transfer member 246, and extension member 220 fixably coupled to member 246 from moving longitudinally relative to base 211; and (b) rotation of handle 243 and barrel 242 in the second direction from the locked position to an “unlocked position” sufficiently decreases compression of block 244 between base 211 and barrel 242 to effectively allow block 244, actuator 241, load transfer member 246, and extension member 220 fixably coupled to member 246 to move longitudinally relative to base 211. In this manner, locking mechanism 240 can be manually controlled by a user via handle 243 to selectively lock and unlock extension member 220 relative to base 211, and thereby selectively lock and unlock the longitudinal position of the corresponding femur support member 230 relative to extension 16 and perineal post 20.

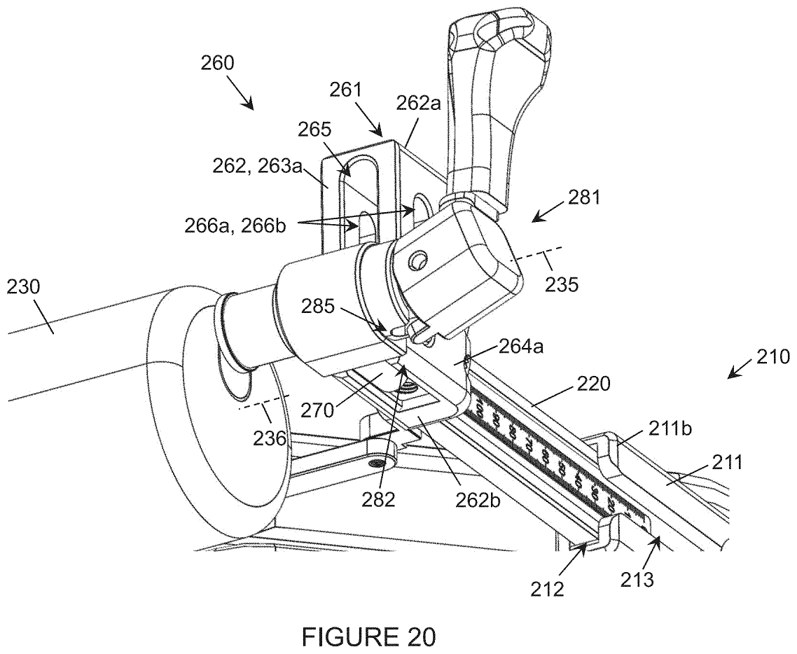

Referring now to FIGS. 20-23, in this embodiment, the vertical position of femur support member 230 relative to a horizontal plane containing the upper surface of base 11 and extension 16 can also be selectively and controllably adjusted by vertical adjustment assembly 260. In this embodiment, vertical adjustment assembly 260 includes a linear vertical adjustment device 261 and a rotational vertical adjustment device 281. As the names imply, linear vertical adjustment assembly 261 moves femur support member 230 linearly up and down to adjust its vertical position, whereas rotational vertical adjustment assembly 281 rotates femur support member 230 to adjust its vertical position. As will be described in more detail below, the vertical movement of femur support member 230 by linear vertical adjustment device 261 results in pure vertical movement of femur support member 230 (i.e., there is no longitudinal or lateral component associated with the vertical movement of femur support member 230), whereas the vertical movement of femur support member 230 by rotational vertical adjustment device 281 does not result in pure vertical movement of femur support member 230 as it induces a longitudinal component of movement in connection with the vertical movement of femur support member 230.

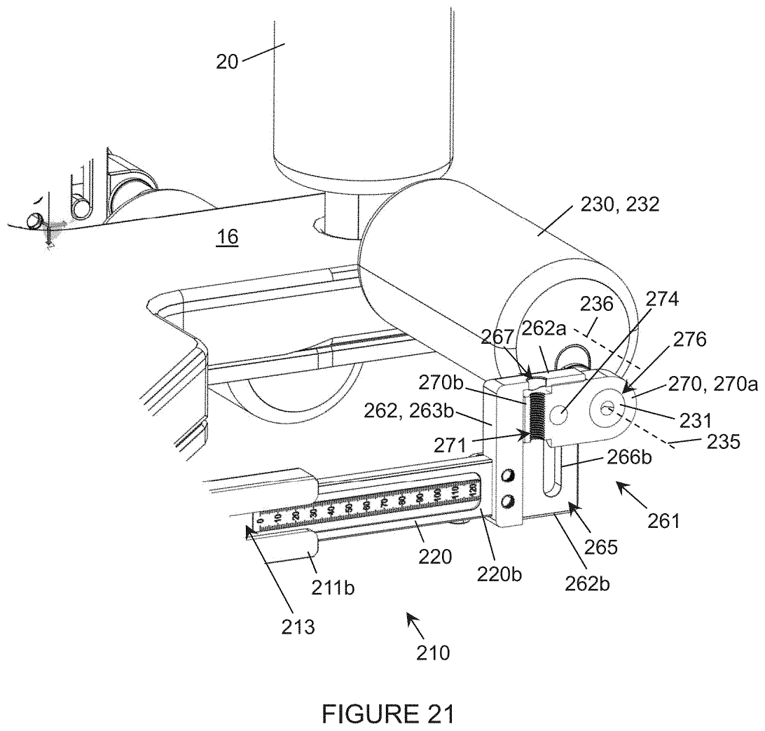

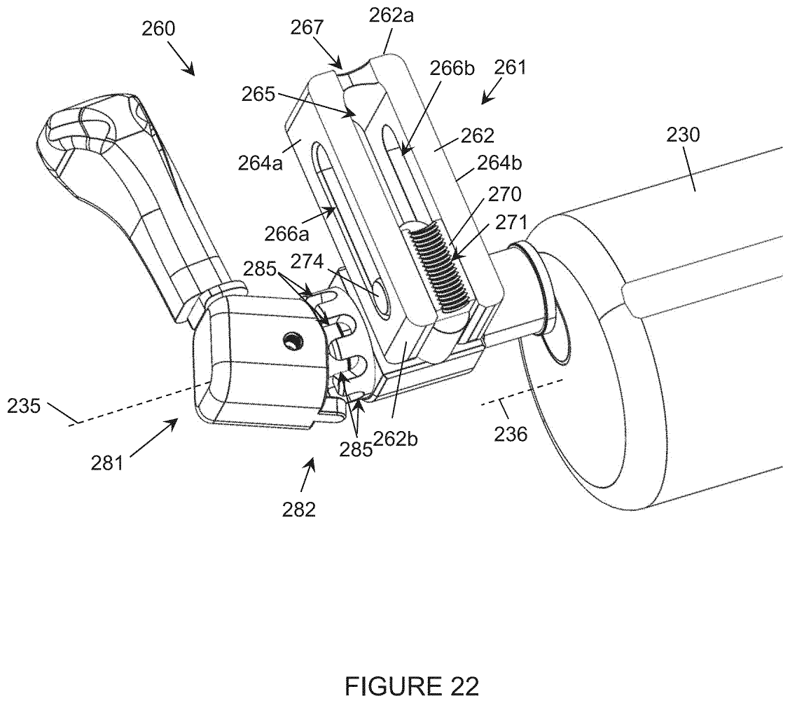

In this embodiment, linear vertical adjustment assembly 261 includes a body 262 fixably attached to end 220b of extension member 220, a slide block 270 moveably coupled to body 262, and an actuator 273 (not shown in FIGS. 21 and 22) configured to selectively and controllably move slide block 270 vertically up and down relative to body 262. In this embodiment, body 262 has a generally rectangular prismatic geometry. In particular, body 262 has an upper end 262a, a lower end 262b, a first or front side 263a extending vertically between ends 262a, 262b, a second or rear side 263b extending vertically between ends 262a, 262b, and a pair of lateral sides 264a, 264b extending vertically between ends 262a, 262b and horizontally between sides 263a, 263b. In addition, body 262 includes an inner cavity 265 extending vertically from lower end 262b toward upper end 262a and extending horizontally from front side 263a toward rear side 263b. Thus, cavity 265 extends through front side 263a and lower end 262b, but does not extend through top side 262a, rear side 263b, or lateral sides 264a, 264b. Body 262 also includes elongate, vertically oriented slots 266a, 266b extending from lateral sides 264a, 264b, respectively, to cavity 265, and a bore 267 extending vertically through upper end 262a to cavity 265. Slots 266a, 266b, cavity 265, and bore 267 extend vertically and are oriented parallel to each other.

Referring still to FIGS. 20-23, slide block 270 is slidably disposed in cavity 265 and extends horizontally from cavity 265 through front side 263a of body 262. In this embodiment, slide block 270 has a generally rectangular prismatic geometry. As best shown in FIG. 21, slide block 270 has a first or front end 270a extending from cavity 265 and external body 262 and a second or rear end 270b disposed within cavity 265 of body 262. Femur support member 230 is rotatably coupled to front end 270a and extends laterally and horizontally inward or medially therefrom. More specifically, a cylindrical throughbore 276 extends horizontally through slide block 270 proximal first end 270a. A post 231 of femur support member 230 is rotatably disposed in throughbore 276, and an annular pad 232 of femur support member 230 is fixably mounted to post 231. However, as will be described in more detail below, post 231 and pad 232 are not coaxially aligned. In addition, slide block 270 includes an internally threaded throughbore 271 extending vertically therethrough and positioned proximal near end 270b. Slide block 270 is sized and positioned such that when slide block 270 is seated in cavity 265 it slidingly engages the inner surfaces of body 262 defining cavity 265 with throughbore 271 vertically and coaxially aligned with bore 267 in body 262. A guide pin 274 extends horizontally through slide block 270 between ends 270a, 270b and into mating slots 266a, 266b of body 262. In particular, the ends of guide pin 274 are slidingly disposed in mating slots 266a, 266b. Sliding engagement of slide block 270 and body 262 and sliding engagement of guide pin 274 and slots 266a, 266b generally limit relative movement of slide block 270 relative to body 262 to the vertical direction (up and down).

As previously described, actuator 273 is used to move slide block 270, and hence move femur support member 230 vertically up and down relative to body 262 and the horizontal plane containing the horizontal upper surface of base 11 and extension 16. In this embodiment, actuator 273 is an externally threaded, vertically oriented bolt, and in particular, a hex head bolt. The head of bolt 273 bears against upper end 262a of body 262 as the shaft of bolt 273 extends vertically through bore 262 and is threaded into mating throughbore 271. To selectively and controllably move slide block 270 vertically upward relative to body 262, and hence, move femur support member 230 upward relative to the horizontal plane containing the horizontal upper surface of base 11 and extension 16, bolt 273 is rotated in a first direction to threadably advance bolt 273 through throughbore 271; and to selectively and controllably move slide block 270 vertically downward relative to body 262, and hence, move femur support member 230 downward relative to the horizontal plane containing the horizontal upper surface of base 11 and extension 16, bolt 273 is rotated in a second direction opposite to the first direction to threadably withdraw bolt 273 from throughbore 271. In general, bolt 273 can be rotated manually or electronically to vertically raise and lower femur support member 230.

Referring now to FIGS. 20-25b, in this embodiment, rotational vertical adjustment device 281 includes elongate post 231 of femur support member 230 rotatably disposed in throughbore 276 of slide block 270 (FIGS. 21, 24b, and 25b) and a locking assembly 282 to selectively and controllably lock the rotational position of post 231 relative to slide block 270. Post 231 has a central axis 235 about which it can be selectively rotated (in either direction) within throughbore 276 relative to slide block 270. As previously described and as best shown in FIGS. 20-22, pad 232 is fixably coupled to post 231 and oriented parallel to post 231, however, pad 232 is not coaxially aligned with axis 235 of post 231. Rather, pad 232 has a horizontally oriented central axis 236 that is radially offset from axis 235. Accordingly, when post 231 is rotated about central axis 235 relative to slide block 270, pad 232 and axis 236 simultaneously orbit about axis 235 and cyclically move vertically up and down.

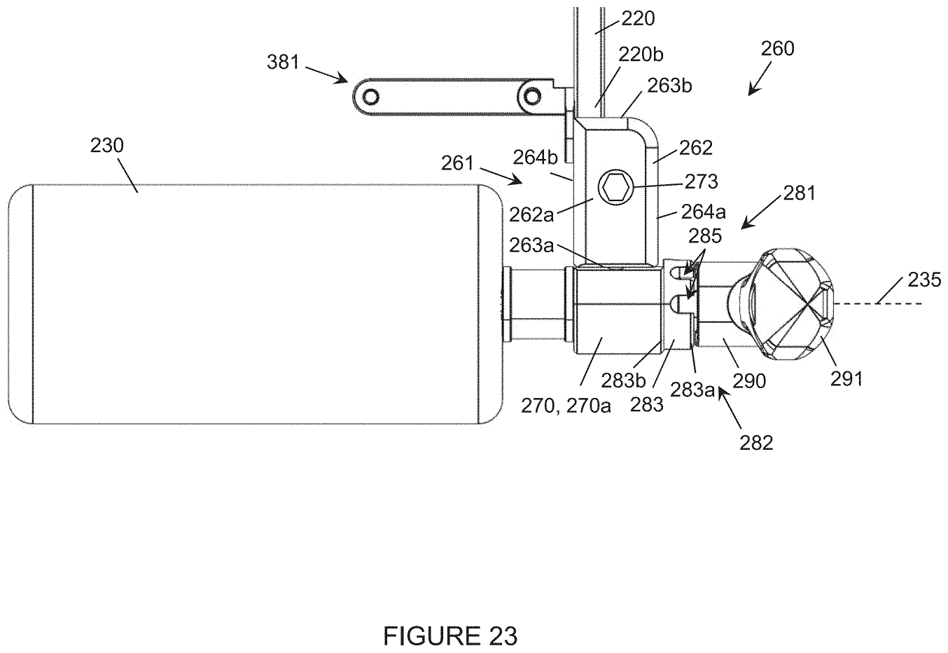

Referring now to FIGS. 23-25b, in this embodiment, locking assembly 282 includes a collar 283 fixably coupled to slide block 270, a locking member 290 pivotally coupled to post 231, and a handle 291 attached to locking member 290. In this embodiment, collar 283 is integral with slide block 270. In general, handle 291 is used to selectively and controllably pivot locking member 290 relative to post 231 to rotationally lock and unlock locking member 290 and post 231 relative to collar 283 and slide block 270.

Collar 283 is disposed about and coaxially aligned with post 231. Thus, collar 283 is horizontally oriented. In particular, collar 283 has a first or laterally outer end 283a, a second or laterally inner end 283b, and a cylindrical throughbore 284 extending horizontally from first end 283a to second end 283b. Throughbore 284 is coaxially aligned and has the same diameter of throughbore 276 in slide block 270, and thus, is effectively an extension of throughbore 276. Post 231 extends through throughbore 284 and slidably engages collar 283. In addition, as best shown in FIGS. 20, 22, and 23, collar 283 includes a plurality of circumferentially spaced recesses or detents 285 disposed along end 283a. In this embodiment, five uniformly circumferentially-spaced detents 285 are provided over half (i.e., 180°) of the circumference of collar 283. In particular, the five detents 285 are angularly spaced 45° apart with a first detent 285 disposed at the top of collar 283, a second detent 285 disposed at the bottom of collar 283 (angularly spaced 180° from the first detent 285), and three detents 285 positioned between the first detent 285 and the second detent 285 along the rearward facing side (proximal side) of collar 283. Thus, with the first detent 285 at the top of collar 283 being disposed at 0° and the second detent 285 at the bottom of collar 283 being disposed at 180°, the remaining three detents 285 are disposed at 45°, 90°, and 135° moving circumferentially from the first detent 285 to the second detent 285 along the rearward facing half of collar 283.

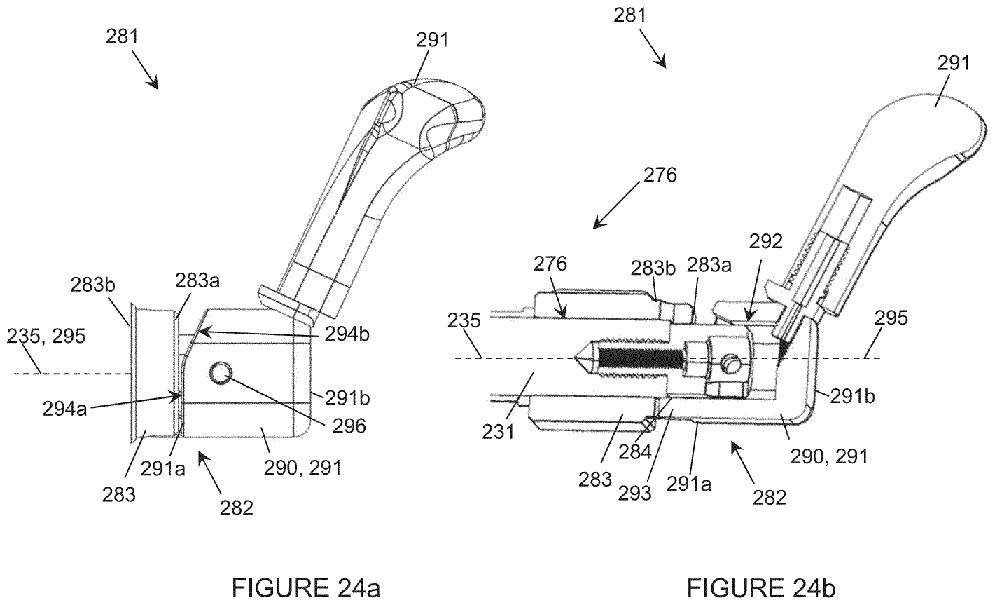

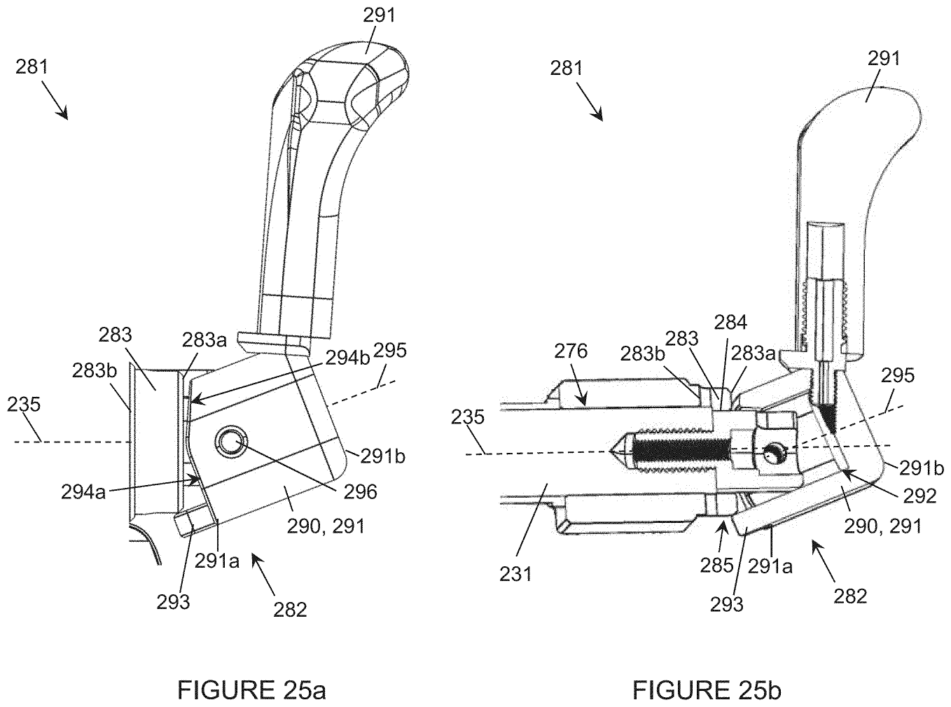

Referring again to FIGS. 24a-25b, locking member 290 has a generally cup-shaped body 291 that is pivotally coupled to the laterally outer end of post 231. More specifically, body 291 has a central axis 295, a first or laterally inner end 291a, a second or laterally outer end 291b, a receptacle 292 extending axially from inner end 291a, and a projection or finger 293 extending from inner end 291a. The laterally outer end of post 231 extends into receptacle 292. Inner end 291a includes a first planar surface 294a disposed in a plane oriented perpendicular to central axis 295 and a second planar surface 294b oriented at an acute angle relative to a plane oriented perpendicular to axis 295. Planar surface 294b extends from planar surface 294a but slopes axially toward outer end 291b in side view moving from planar surface 294a. Each surface 294a, 294b extends circumferentially about 180° about axis 295. Projection 293 extends axially from first planar surface 294a at end 291a. As will be described in more detail below, projection 293 is sized to mate and removably engage with detents 285.

Referring still to FIGS. 24a-25b, as previously described, locking member 290 is pivotally coupled to the laterally outer end of post 231. In this embodiment, a cylindrical pin 296 (not shown in FIGS. 24b and 25b) extends radially through locking member 290 and post 231. Thus, locking member 290 and post 231 rotate together about the central axis 235 of post 231 but locking member 290 can pivot relative to post 231 about pin 296. Clearances are provided between (i) the outer end of post 231 and the inner surfaces of body 291 defining receptacle 292, and (ii) second planar surface 294b and the axially adjacent laterally outer end 283a of collar 283 to allow locking member 290 to pivot about pin 296 relative to the laterally outer end of post 231. Handle 291 is fixably attached to body 291 of locking member 290 and can be used to pivot locking member 290 about pin 296 relative to post 231.

When projection 293 is aligned with any particular detent 285, projection 293 can be moved in and out of positive engagement with that detent 285 by pivoting locking member 290 about pin 296. As best shown in FIGS. 24a and 24b, when projection 293 is seated in a detent 285, engagement of projection 293 and that detent 285 prevents locking member 290, and hence post 231, from rotating relative to collar 283 and slide block 270. However, as best shown in FIGS. 25a, and 25b, when projection 293 is not disposed in a detent 285, locking member 290, and hence post 231, can rotate relative to collar 283 and slide block 270. Thus, projection 293, locking member 290, and locking assembly 282 may each be described as having a “locked position” with projection 293 seated within a detent 285, thereby preventing rotation of post 231 relative to collar 283 and slide block 270; and an “unlocked” position with projection 293 withdrawn from detents 285, thereby allowing rotation of post 231 relative to collar 283 and slide block 270. It should be appreciated that handle 291 can be used to both pivot locking member 290 about pin 296 between the locked and unlocked positions, and rotate locking member 290 and post 231. In this embodiment, projection 293, locking member 290, and locking assembly 282 are biased to the locked position by a torsional spring (not shown) disposed about pin 296.

Projection 293 can be moved out of detents 285 by pivoting locking member 290 to the unlocked position, and then locking member 290 can be rotated (along with post 231) to align projection 293 with the desired detent 285 and then pivoted to the locked position to advance projection 293 into that desired detent 285. As previously described, in this embodiment, five detents 285 are disposed along 180° of the rearward facing side (proximal side) of collar 283, and are angularly spaced 45° apart. Thus, locking member 290 and post 231 can generally be rotationally fixed in 45° increments when locking member 290 is rotated to move projection 293 along the rear side of collar 283.

Referring now to FIG. 22, as previously described, post 231 and annular pad 232 are oriented parallel to each other (both are horizontally oriented), but are not coaxially aligned. Accordingly, when post 231 is rotated about central axis 235 relative to slide block 270, pad 232 and axis 236 simultaneously orbit about axis 235 and cyclically move vertically up and down. Accordingly, annular pad 232 can be moved vertically upward by pivoting locking member 290 and projection 293 to the unlocked position, and then rotating projection 293 upward to a vertically higher detent 285; and annular pad 232 can be moved vertically downward by pivoting locking member 290 and projection 293 to the unlocked position, and then rotating projection 293 downward to a vertically lower detent 285. In addition, the vertical position of pad 232 can be fixed and locked at a desired vertical position by aligning projection 293 with the corresponding detent 285 and then transitioning locking member 290 and projection 293 to the locked position.

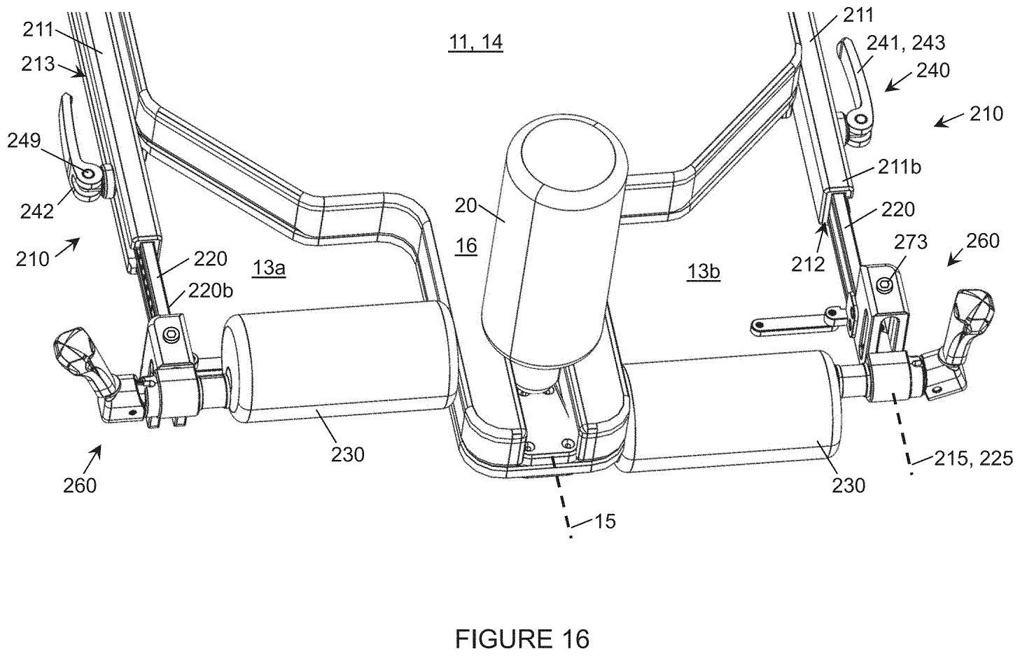

In general, locking member 290 and annular pad 232 can be coupled to post 231 such that (i) annular pad 232 is disposed at its lowermost vertical position when projection 293 is aligned and/or disposed in detent 285 located on the bottom of collar 283 (i.e., the lowermost detent 285), and annular pad 232 disposed at its uppermost vertical position when projection 293 is aligned and/or disposed in detent 285 located on the top of collar 283 (i.e., the uppermost detent 285) as shown on telescopic femur support assembly 210 on the left in FIG. 16; or (ii) annular pad 232 is disposed at its uppermost vertical position when projection 293 is aligned and/or disposed in detent 285 located on the bottom of collar 283 (i.e., the lowermost detent 285), and annular pad 232 disposed at its lowermost vertical position when projection 293 is aligned and/or disposed in detent 285 located on the top of collar 283 (i.e., the uppermost detent 285) as shown on telescopic femur support assembly 210 on the right in FIG. 16.

As described above, locking member 290 and projection 293 are biased to the locked position, and thus, unless force is continuously applied to handle 291 to maintain locking member 290 and projection 293 in the unlocked positions, locking member 290 and projection 293 will automatically transition into the locked positions as projection 293 moves into alignment with a detent 285. Thus, if force is not continuously applied to handle 291 to maintain locking member 290 and projection 293 in the unlocked positions while projection 293 is rotated along the rearward facing side (i.e., proximal side) of collar 283, projection 293 will move into engagement with each successive detent 285 every 45°, thereby allowing selective, controlled, and uniform incremental adjustment of the vertical position of annular pad 232. However, as the forward facing side (i.e., distal side) of collar 283 does not include any detents 285, locking member 290 and projection 293 will remain in the unlocked positions without the need to maintain locking member 290 and projection 293 in the unlocked positions via the continuous application of force to handle 291. This feature provides the user a relatively simple and easy way to rotate post 231 through 180°.

Referring again to FIG. 15, with the patient disposed on base 11 as described above, femur support members 230 can be independently moved longitudinally along the bottom of the patient's thigh to support the corresponding femurs of the patient at the desired and/or optimal longitudinal positions. Each telescopic femur support assembly 210 is configured such that the corresponding extension member 220 and femur support member 230 has the same longitudinal range of motion Lₘ, proximal range of motion Lₚ, and distal range of motion L_{d} as previously described. However, in this embodiment, axes 235, 236 of post 231 and the corresponding annular pad 232, respectively, are not coincident, and thus, the proximal range of motion Lₚ and distal range of motion L_{d} are measured longitudinally from perineal post 20 and reference axis 35 to the central axis 236 of the corresponding annular pad 232. To accommodate most patients of different sizes and anatomies, in embodiments described herein, each telescopic femur support assembly (e.g., each telescopic femur support assembly 210) is sized and arranged such that the longitudinal range of motion Lₘ of each femur support member 230 ranges from 100 mm to 165 mm; the proximal range of motion Lₚ ranges from 0 mm to 120 mm, alternatively ranges from 0 to 110 mm, and alternatively from 0 to 100 mm; and the distal range L_{d} of motion ranges from 0 mm to 45 mm, alternatively ranges from 0 mm to 20 mm, and alternatively is 0 mm. Stated differently, and with longitudinal distances measured proximally from perineal post 20 and reference axis 35 being expressed as positive distances and longitudinal distances measured distally from perineal post 20 and reference axis 35 being expressed in the negative, in embodiments described herein, the longitudinal range of motion Lₘ of each femur support member ranges from −45 mm to 120 mm, alternatively ranges from −20 mm to 110 mm, and alternatively ranges from 0 mm to 100 mm. In addition, and referring now to FIG. 17, each telescopic femur support assembly 210 is configured to have the same vertical range of motion Vₘ, anterior range of motion Vₐ, and a posterior range of motion Vₚ as previously described. To accommodate most patients of different sizes and anatomies, in embodiments described herein, each telescopic femur support assembly 210 is sized and arranged such that the vertical range of motion Vₘ of each femur support member 230 ranges from 40 mm to 130 mm; the anterior range of motion ranges from 0 mm to 80 mm, alternatively ranges from 0 mm to 60 mm, and alternatively ranges from 0 mm to 40 mm; and the posterior range of motion ranges from 0 mm to 50 mm, alternatively ranges from 0 mm to 25 mm, and alternatively is 0 mm. Stated differently, and with vertical distances measured upward and anteriorly from the horizontal plane containing upper surface of extension 16 and base 11 being expressed as positive distances and vertical distances measured downward and posteriorly from the horizontal plane containing upper surface of extension 16 and base 11 being expressed in the negative, in embodiments described herein, the vertical range of motion of each femur support member ranges from −50 mm to 80 mm, alternatively ranges from −25 mm to 60 mm, and alternatively ranges from 0 mm to 40 mm. During surgery or diagnostic procedure on a patient's operative leg, it may be desirable to image the bones (e.g., femur, pelvis, etc.) or other anatomy of the patient. Accordingly, embodiments of systems for directly supporting a patient's thigh(s) described herein described herein (e.g., systems 10, 10′, 10″, 200) can be adapted to allow for imaging, and in particular, to accommodate imaging cassettes (e.g., X-ray imaging cassettes) for obtaining such images. In general, embodiments described herein can include structures for removably holding and supporting one or more imaging cassettes in positions immediately below the table 11, and more specifically, immediately below recesses 13a, 13b, extension 16, and femur support members 120, 230.

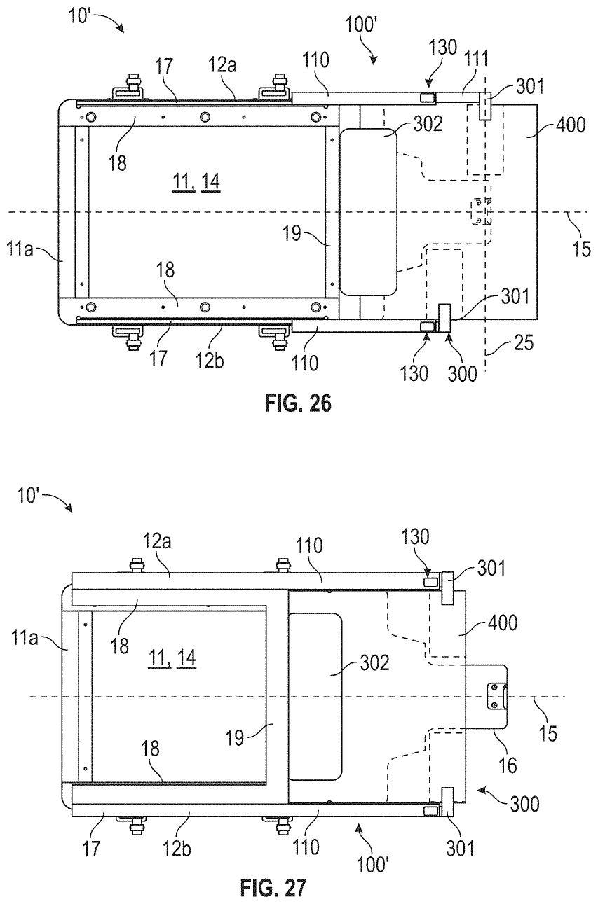

FIGS. 23-35 illustrate embodiments of support assemblies for accommodating and releasably holding an imaging cassette in a static position immediately below recesses 13a, 13b, extension 16, and femur support members 120. Typically, an imaging cassette such as an X-ray cassette is a flat, rectangular structure that holds film or sensors that respond to radiation (e.g., X-rays) directed into and passing through the region of interest in the patient (i.e., the region of the patient to be imaged) to generate an image. In embodiments described herein, tubular bases 110 and extension members 111 are sized and positioned such that the lateral distance therebetween (measured perpendicular to longitudinal axis 15) is slightly greater than the width of the imaging cassette such that the imaging cassette can be slid and positioned directly between tubular bases 110 and extension members 111. In FIGS. 26-38, the imaging cassettes are designated with reference numeral 400. Although FIGS. 26-38 illustrate embodiments support assemblies for accommodating and releasably holding imaging cassettes in connection with system 10′ previously described, it should be appreciated that the embodiments shown in FIGS. 26-38 can also be used in connection with any of the embodiments for directly supporting a patient's thigh(s) described herein including systems 10, 10′, 10″, 200.

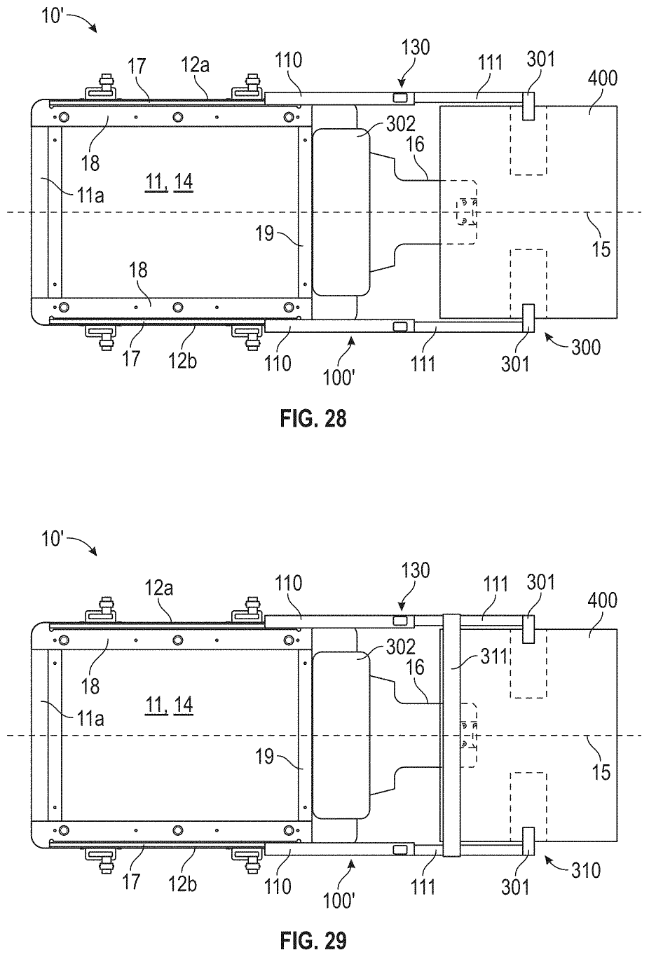

Referring first to FIGS. 26-28, an embodiment of a support assembly 300 for accommodating and releasably holding an imaging cassette 400 is shown. In this embodiment, support assembly 300 includes a pair of support tabs 301 and a rectangular support shelf 302. Tabs 301 are mounted to the bottom of extension members 111 and extend laterally inward from the corresponding extension member 111 toward longitudinal axis 15. Shelf 302 is mounted to the bottom of table 11 at the intersection of portion 14 and extension 16, and extends into recesses 13a, 13b in bottom view. As shown in FIGS. 26 and 27, imaging cassette 400 can be slid below extension 16 and onto tabs 301 and shelf 302, which support imaging cassette 400 from below. Tabs 301 can move independently, with each tab 301 moving with the corresponding extension member 111 but still providing support to imaging cassette 400. Consequently, as shown in FIG. 28, imaging cassette 400 can be slid below extension 16 and onto tabs 301 (but not onto shelf 302), which support imaging cassette 400 from below. This enables imaging cassette 400 to be positioned immediately below recesses 13a, 13b and femur support members 120 when femur support members 120 are positioned beyond perineal post 20.

Referring now to FIG. 29, an embodiment of a support assembly 310 for accommodating and releasably holding an imaging cassette 400 is shown. Support assembly 310 is similar to support assembly 300 previously described. In particular, in this embodiment, support assembly 310 includes tabs 301 as previously described, shelf 302 as previously described, and a support bar 311 extending laterally across recesses 13a, 13b between extension members 111. Support bar 321 can be removably mounted to the bottoms of extension members 111 when femur support members 120 are in the desired positions to provide additional support to imaging cassette 400 (whether it can or cannot be seated on shelf 302). Imaging cassette 400 is slid below extension 16 and onto tabs 301 and support bar 311, which support imaging cassette 400 from below.

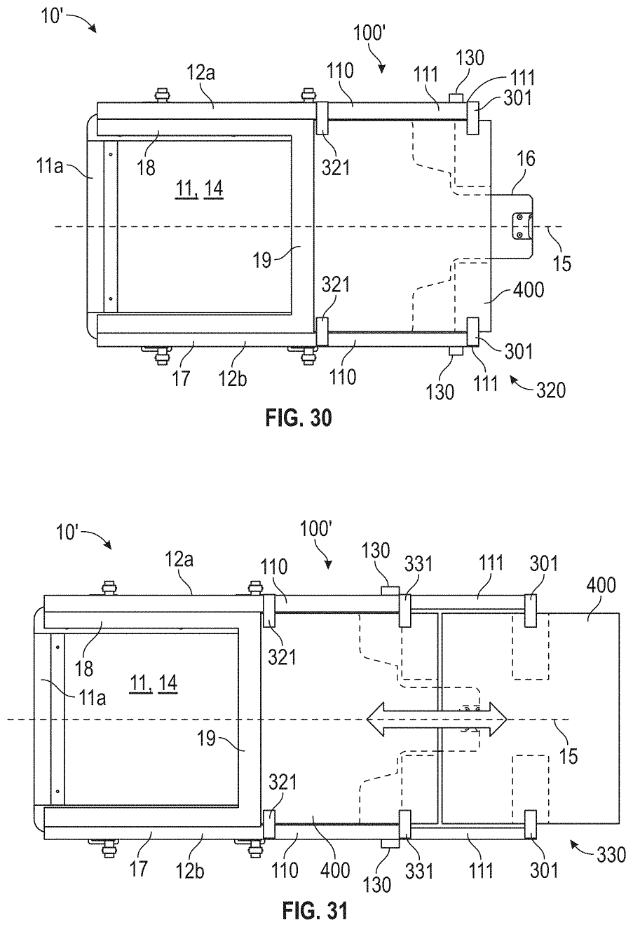

Referring now to FIG. 30, an embodiment of a support assembly 320 for accommodating and releasably holding an imaging cassette 400 is shown. Support assembly 320 is similar to support assembly 300 previously described. In particular, in this embodiment, support assembly 320 includes tabs 301 as previously described, but instead of shelf 302, support assembly 320 includes an additional pair of support tabs 321. Tabs 321 are mounted to the bottom of tubular bases 110 adjacent portion 14 and extend laterally inward from the corresponding tubular base 110 toward longitudinal axis 15. Imaging cassette 400 is slid below extension 16 and onto tabs 301, 311, which support imaging cassette 400 from below. As previously described, tabs 301 can move independently, with each tab 301 moving with the corresponding extension member 111 but still providing support to imaging cassette 400. This embodiment would allow the imaging cassette 400 to be slid proximally under base 11, so that the distal end of the imaging cassette 400 will not extend distally past the femoral support members 120.

Referring now to FIG. 31, an embodiment of a support assembly 330 for accommodating and releasably holding an imaging cassette 400 is shown. Support assembly 330 is similar to support assembly 320 previously described. In particular, in this embodiment, support assembly 330 includes tabs 301, 321 as previously described mounted to tubular bases 110 and extension members 111. In addition, in this embodiment, support assembly 330 includes a pair of tabs 331 mounted to the bottom of tubular bases 110 distal portion 14 and extend laterally inward from the corresponding tubular base 110 toward longitudinal axis 15. Imaging cassette 400 is slid below extension 16 and onto tabs 301, 311, 331, which support imaging cassette 400 from below. As previously described, tabs 301 can move independently, with each tab 301 moving with the corresponding extension member 111 but still providing support to imaging cassette 400. As shown in FIG. 31, with femur support members 120 extended beyond perineal post 20, the longitudinal spacing of tabs 301, 311, 331 enables tabs 301, 311, 331 to accommodate and releasably hold an imaging cassette 400 that can be positioned in either position shown in FIG. 31 or any longitudinal position between the positions shown in FIG. 31.

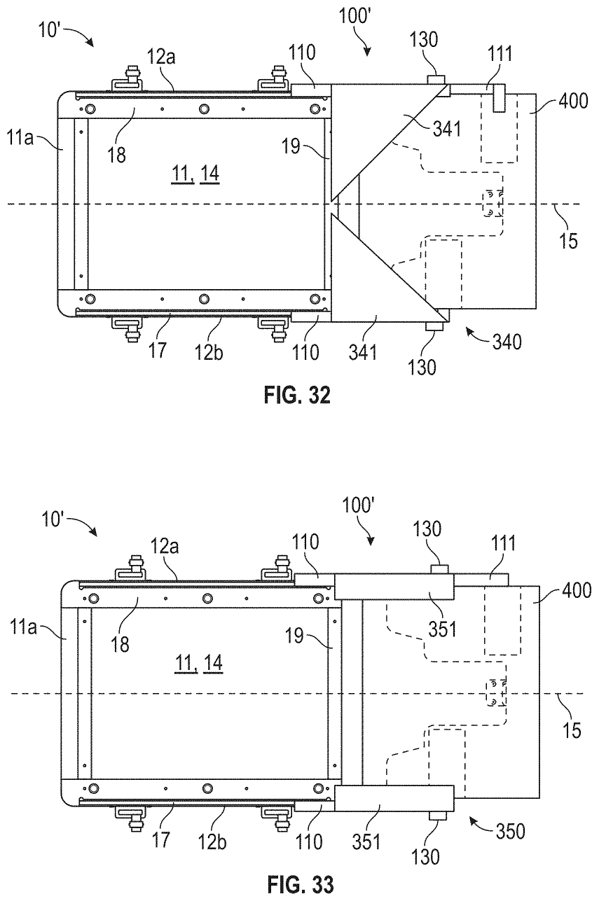

Referring now to FIG. 32, an embodiment of a support assembly 340 for accommodating and releasably holding an imaging cassette 400 is shown. In this embodiment, support assembly 340 includes a pair of triangular support shelves 341 mounted to the bottom of tubular bases 110 and the bottom of one cross-member 19 below portion 14. Shelves 341 extend below the corners of recesses 13a, 13b generally positioned at the intersections of tubular bases 110 and portion 14 in bottom view. Imaging cassette 400 is slid below extension 16 and onto shelves 341, which support imaging cassette 400 from below.

Referring now to FIG. 33, an embodiment of a support assembly 350 for accommodating and releasably holding an imaging cassette 400 is shown. In this embodiment, support assembly 350 includes a pair of elongate rectangular shelves 351 mounted to the bottom of tubular bases 110. Shelves 351 extend below the laterally outer portions of recesses 13a, 13b laterally adjacent bases 110 and proximal portion 14 in bottom view. As shown in FIG. 33, shelves 351 do not extend longitudinally below extension members 111. Imaging cassette 400 is slid below extension 16 and onto shelves 351, which support imaging cassette 400 from below.

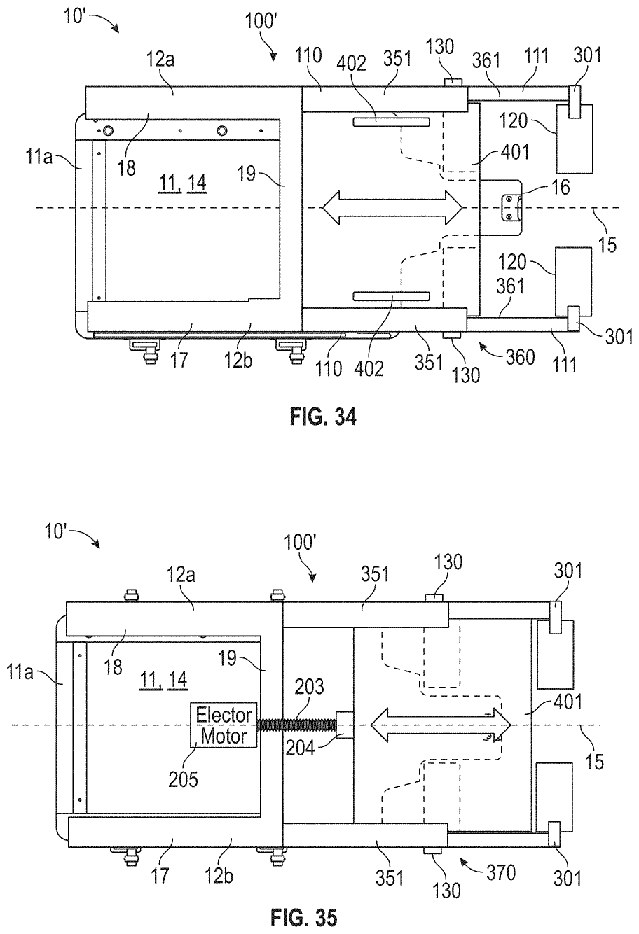

Referring now to FIG. 34, an embodiment of a support assembly 360 for accommodating and releasably holding an imaging cassette tray 401 is shown. Imaging cassette tray 401 can accommodate and support an imaging cassette 400. Support assembly 360 is similar to support assembly 350 previously described. In particular, support assembly 350 includes a pair of elongate rectangular shelves 351 as previously described that slidingly support imaging cassette tray 401. An imaging cassette 400 is slid below extension 16 and onto imaging cassette tray 401, which has handles 402 extending downward to allow adjustment of the longitudinal position of the imaging cassette tray 401 and imaging cassette 400 disposed thereon.

Referring now to FIG. 35, an embodiment of a support assembly 370 for accommodating and releasably holding an imaging cassette 400 is shown. Support assembly 370 is similar to support assembly 360 previously described. In particular, support assembly 370 includes a pair of elongate rectangular shelves 351 as previously described that support an imaging cassette tray 401. Imaging cassette 400 is slid below extension 16 and onto imaging cassette tray 401, which supports imaging cassette 400 from below. However, in this embodiment, support assembly 370 also includes an electric motor 205 mounted under base 11 and a threaded rod 203 that can be controllably rotated by motor 205 and engages mating internal threads within motor 205. Rod 203 extends through cross-member 19 and rotatably engages an adapter 204 coupled to imaging cassette tray 401. Thus, by rotating threaded rod 203 with motor 205, rod 203 can be extended and retracted relative to motor 205, thereby moving adjusting the longitudinal position of imaging cassette tray 401 and imaging cassette 400 disposed thereon.

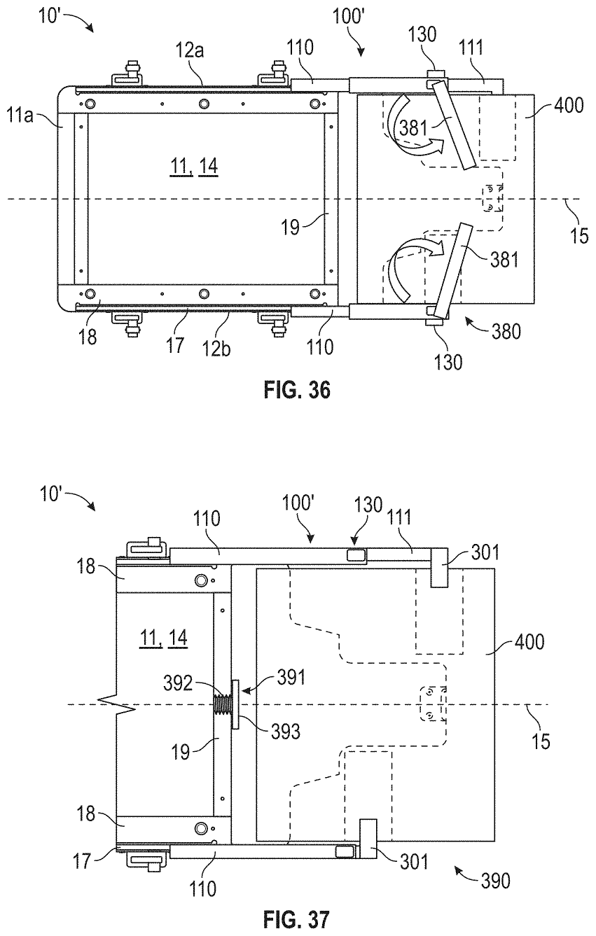

Referring now to FIG. 36, an embodiment of a support assembly 380 for accommodating and releasably holding an imaging cassette 400 is shown. In this embodiment, support assembly 380 includes a pair of elongate rectangular arms 381 pivotally mounted to the bottom of tubular bases 110. Arms 381 can be rotated in a horizontal plane between a stowed position generally retracted from below recesses 13a, 13b and extending along the bottom of bases 110 and a deployed position generally extending laterally from bases 110 into and below recesses 13a, 13b in bottom view. Imaging cassette 400 is slid below extension 16 and onto arms 381 with arms 381 in the deployed position to support imaging cassette 400 from below. Arms 381 can also be mounted to extension members 111 as shown in FIGS. 16 and 23.

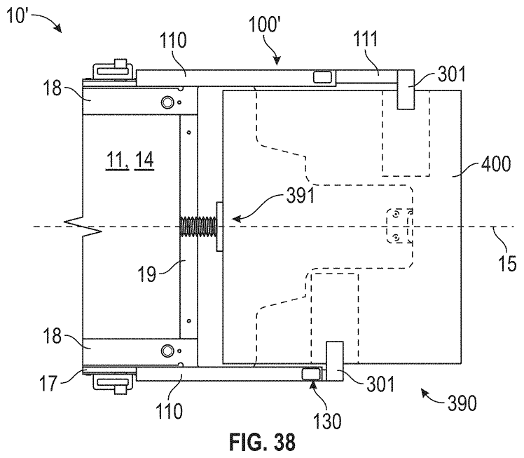

Referring now to FIGS. 37 and 38, an embodiment of a support assembly 390 for accommodating and releasably holding an imaging cassette 400 is shown. In this embodiment, support assembly 390 includes a pair of support tabs 301 as previously described and a depth adjustment device 391 mounted to a cross-member 19 adjacent the intersection between portion 14 and extension 16. Depth adjustment device 391 can be actuated to adjust the axial position (relative to longitudinal axis 15) of imaging cassette 400 relative to base 11, extension 16, and recesses 13a, 13b. In this embodiment, adjustment device 391 includes a threaded shaft 392 and a bumper plate 393 mounted to an end of shaft 392. Shaft 392 extends through cross-member 19 and engages mating threads in cross-member 19. Thus, by rotating shaft 392, the axial position of bumper plate 393 can be adjusted. Imaging cassette 400 is slid below extension 16 and onto tabs 301 unto it axially abuts bumper plate 393.

While preferred embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. For example, the relative dimensions of various parts, the materials from which the various parts are made, and other parameters can be varied. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

What is claimed is:

1. A telescopic assembly for supporting a patient's femur, the assembly comprising:
an elongate tubular base configured to be fixably attached to a table, wherein the tubular base has a longitudinal axis;
an elongate extension member slidably disposed in the tubular base and configured to telescopically extend longitudinally from the tubular base;
a femur support member coupled to an end of the extension member and configured to be positioned below and support the femur of the patient, wherein the femur support member is configured to move longitudinally with the extension member relative to the tubular base.

2. The telescopic assembly of claim 1, wherein the femur support member has a longitudinal range of motion that ranges from 100 mm to 165 mm.

3. The telescopic assembly of claim 2, further comprising a first locking mechanism configured to releasably lock the extension member relative to the tubular base.

4. The telescopic assembly of claim 3, wherein the first locking mechanism is configured to releasably lock the extension member at a plurality of incremental longitudinal positions relative to the tubular base.

5. The telescopic assembly of claim 3, wherein the femur support member is configured to move vertically relative to the extension member.

6. The telescopic assembly of claim 5, wherein the femur support member has a vertical range of motion that ranges from 40 mm to 130 mm.

7. The telescopic assembly of claim 6, further comprising a tubular sleeve fixably attached to an end of the extension member distal the tubular base and an extension post slidably disposed in the tubular sleeve, wherein the femur support member is fixably attached to an upper end of the extension post.

8. The telescopic assembly of claim 7, further comprising a second locking mechanism configured to releasably lock the extension post relative to the tubular sleeve.

9. The telescopic assembly of claim 8, wherein the second locking mechanism is configured to releasably lock the extension post at a plurality of incremental vertical positions relative to the tubular sleeve.

10. The telescopic assembly of claim 5, wherein the femur support member is configured to move vertically relative to the extension member independent of being moved longitudinally relative to the tubular base.

11. A system for supporting a thigh and femur of a patient during a surgical or diagnostic procedure, the system comprising:
a table having a longitudinal axis, a first end, a second end, and a pair of lateral sides extending between the first end and the second end, wherein the table comprises:
a pair of laterally spaced recesses at the intersections of the lateral sides and the second end;
a base extending from the first end to the recesses;
a horizontal extension positioned between the recesses and extending longitudinally from the base to the second end; and
a perineal post extending vertically from the extension at the second end of the table;
a telescopic assembly coupled to the table along one of the lateral sides of the table, wherein the telescopic assembly comprises:
an elongate tubular base fixably attached to the table, wherein the tubular base has a longitudinal axis oriented parallel to the longitudinal axis of the table;
an extension member slidably disposed in the tubular base and configured to telescopically extend longitudinally from the tubular base;
a femur support member coupled to an end of the extension member distal the tubular base and configured to support the thigh and femur of the patient, wherein the femur support member is configured to move longitudinally with the extension member relative to the tubular base.

12. The system of claim 11, wherein the femur support member has a longitudinal range of motion that ranges from 100 mm to 165 mm.

13. The system of claim 12, wherein the femur support member has a proximal range of motion measured longitudinally from the perineal post toward the base of the table and a distal range of motion measured longitudinally from the perineal post away the base of the table;
wherein the proximal range of motion ranges from 0 mm to 120 mm and the distal range of motion ranges from 0 mm to 45 mm.

14. The system of claim 12, wherein the femur support member is configured to move vertically relative to the extension member.

15. The system of claim 14, wherein the femur support member has a vertical range of motion that ranges from 40 mm to 130 mm.

16. The system of claim 15, wherein the femur support member has an anterior range of motion measured vertically upward from a horizontal reference plane containing an upper surface of the base of the table to an uppermost peak of the femur support member, and wherein the femur support member has a posterior range of motion measured vertically downward from the horizontal reference plane to the uppermost peak of the femur support member;
wherein the anterior range of motion ranges from 0 mm to 80 mm; and
wherein the posterior range of motion ranges from 0 mm to 50 mm.

17. The system of claim 14, further comprising:
a first locking mechanism configured to releasably lock a longitudinal position of the femur support member relative to the tubular base;
a second locking mechanism configured to releasably lock a vertical position of the femur support member relative to the tubular base.

18. The system of claim 17, wherein the femur support member is configured to move longitudinally and vertically independent of each other.

19. The system of claim 11, further comprising means for supporting an imaging cassette below the femur support member and the perineal post.
